(19) United States
(12) Patent Application Publication
(10) Pub. No.: US 2025/0154212 A1
(43) Pub. Date: May 15, 2025

(54) METHOD OF DISRUPTING MEMORY AND LIPOPEPTIDE FOR USE IN SUCH METHOD

(71) Applicant: The Research Foundation for The State University of New York, Albany, NY (US)

(72) Inventor: Todd C. SACKTOR, Albany, NY (US)

(73) Assignee: The Research Foundation for The State University of New York, Albany, NY (US)

(21) Appl. No.: 18/941,696

(22) Filed: Nov. 8, 2024

Related U.S. Application Data
(60) Provisional application No. 63/597,518, filed on Nov. 9, 2023.

(51) Int. Cl. C07K 14/47; A61K 38/00; A61P 25/00

(52) U.S. Cl. CPC C07K 14/47; A61P 25/00; A61K 38/00

(57) ABSTRACT

Provided is a method of disrupting memory, including administering to a subject a peptide, wherein the peptide prevents biding of kidney and brain expressed protein to protein kinase M zeta. Administering may include administering to a subject in need of treatment for an addition, neuropathic pain, or an anxiety disorder. The peptide may include amino acid sequence of SEQ ID NO: 1, SEQ ID NO: 2, SEQ ID NO: 3, SEQ ID NO: 4, SEQ ID NO: 5, OR SEQ ID NO: 6. Also provided is a lipopeptide including the amino acid sequence and a fatty acyl group selected from a C12 to a C18 fatty acid.

### CROSS-REFERENCE TO RELATED APPLICATIONS

This application claims benefit of priority from U.S. Provisional Patent Application No. 63/597,518, filed Nov. 9, 2023, the entire content of which is incorporated herein by reference.

### SEQUENCE LISTING

The instant application contains an electronic sequence listing. The contents of the electronic sequence listing T100-2295 Sequence Listing 0097.206A.xml; Size: 29,920 bytes; and Date of Creation: Nov. 4, 2024, is herein incorporated by reference in its entirety.

### BACKGROUND

How molecules lasting only hours to days can maintain memory that persists weeks to years is a long-standing fundamental question in neuroscience (1, 2). LTP is widely considered a putative physiological substrate of memory because strong afferent synaptic stimulation persistently potentiates only activated synaptic pathways; unstimulated pathways remain unchanged (4). Thus, the molecular interaction that maintains synaptic enhancement might also continually target the action of potentiating molecules to activated synapses. Progress towards elucidating this mechanism has been slow, however, because the molecules potentiating synaptic transmission during late-LTP and memory maintenance have not been clearly established.

Further, several maladaptive conscious, mental, cognitive, emotional, sensory, and mental states are believed to result at least in part from aberrant overactivity or disadvantageously persistent activity of neural circuits, which activity may result from or be sustained a process akin to those engaged by adaptive memory and LTP functions. Increasing evidence suggests that interventions capable of disrupting such processes that usually function to promote memory but become co-opted by pathophysiological neural states would be useful when brought to bear in treating conditions such as fear, anxiety, depression, addiction, neuropathic pain, and the like. However, effectiveness and availability of such interventions for clinical use remain limited

The present disclosure is directed to overcoming these and other deficiencies in the art.

### SUMMARY

In an aspect, provided is a method of disrupting memory, including administering to a subject a peptide, wherein the peptide prevents biding of kidney and brain expressed protein (KIBRA) to protein kinase M zeta (PKMzeta), and the peptide includes amino acid sequence X₉₅₈X₉₅₉X₉₆₀X₉₆₁X₉₆₂X₉₆₃X₉₆₄X₉₆₅X₉₆₆X₉₆₇X₉₆₈X₉₆₉X₉₇₀, wherein X₉₅₈ through X₉₇₀ include acids amino acids FVRNSLERRSVRM, respectively, except that one or more of X₉₅₈ may be any amino acid other than F, X₉₅₉ may be any amino acid other than V, X₉₆₀ may be any amino acid other than R, X₉₆₁ may be any amino acid other than N, X₉₆₂ may be any amino acid other than S, X₉₆₃ may be any amino acid other than L, X₉₆₄ may be any amino acid other than E, X₉₆₆ may be any amino acid other than R, X₉₆₇ may be any amino acid other than S, X₉₆₈ may be any amino acid other than V, X₉₆₉ may be any amino acid other than R, and X₉₇₀ may be any amino acid other than M.

Any one or more of X₉₅₈ may be A, X₉₅₉ may be A, X₉₆₀ may be A, X₉₆₁ may be A, X₉₆₂ may be A, X₉₆₃ may be A, X₉₆₄ may be A, X₉₆₆ may be A, X₉₆₇ may be A, X₉₆₈ may be A, X₉₆₉ may be A, and X₉₇₀ may be A, X₉₅₈ may be A, X₉₅₉ may be A, X₉₆₀ may be A, X₉₆₁ may be A, X₉₆₂ may be A, X₉₆₃ may be A, X₉₆₄ may be A, X₉₆₆ may be A, X₉₆₇ may be A, X₉₆₈ may be A, X₉₆₉ may be A, or X₉₇₀ may be A.

The amino acid sequence may include X₉₅₆X₉₅₇X₉₅₈FVRNSLERRSVRM, wherein X₉₅₆ may be A or any amino acid other than P, or X₉₅₇ may be A or any amino acid other than P. The amino acid sequence may include DSSTLSKKX₉₅₆X₉₅₇X₉₅₈X₉₅₉X₉₆₀X₉₆₁X₉₆₂X₉₆₃X₉₆₄X₉₆₅X₉₆₆X₉₆₇X₉₆₈X₉₆₉X₉₇₀KRPSPPPQ.

The amino acid sequence may be selected from SEQ ID NO: 1, SEQ ID NO: 2, SEQ ID NO: 3, 4 SEQ ID NO: 4, and SEQ ID NO: 5. The peptide may further include a cell-penetrating peptide sequence. The cell-penetrating peptide may be selected from SEQ ID NO: 27, SEQ ID NO: 28, SEQ ID NO: 29, and SEQ ID NO: 30. The peptide may further include a fatty acyl group, wherein the fatty acyl group is from a C12 to a C18 fatty acid. The peptide may include N-terminal myristoylation. The administering may include administering a vector and the vector may contain the peptide. The administering may include intracerebral injection. The administering may be selected from septohippocampal administration, amygdalar administration, cerebral cortical administration, spinal cord administration, striatal administration, and cerebellar administration.

The administering may include administering to a subject in need of treatment for one or more of an addition, neuropathic pain, and an anxiety disorder.

The amino acid sequence may be SEQ ID NO: 1. The amino acid sequence may be SEQ ID NO: 2. The amino acid sequence may be SEQ ID NO: 3. The amino acid sequence may be SEQ ID NO: 4. The amino acid sequence may be SEQ ID NO: 5.

The administering may include administering to a subject in need of treatment for an addition. The administering may include administering to a subject in need of treatment for neuropathic pain. The administering may include administering to a subject in need of treatment for an anxiety disorder.

In an aspect, provided is a peptide, wherein the peptide prevents biding of kidney and brain expressed protein (KIBRA) to protein kinase M zeta (PKMzeta), the peptide comprises amino acid sequence X₉₅₈X₉₅₉X₉₆₀X₉₆₁X₉₆₂X₉₆₃X₉₆₄X₉₆₅X₉₆₆X₉₆₇X₉₆₈X₉₆₉X₉₇₀, wherein X₉₅₈ through X₉₇₀ comprise acids amino acids FVRNSLERRSVRM, respectively, except that X₉₅₈ may be any amino acid other than F, X₉₅₉ may be any amino acid other than V, X₉₆₀ may be any amino acid other than R, X₉₆₁ may be any amino acid other than N, X₉₆₂ may be any amino acid other than S, X₉₆₃ may be any amino acid other than L, X₉₆₄ may be any amino acid other than E, X₉₆₅ is R, X₉₆₆ may be any amino acid other than R, X₉₆₇ may be any amino acid other than S, X₉₆₈ may be any amino acid other than V, X₉₆₉ may be any amino acid other than R, or X₉₇₀ may be any amino acid other than M, and wherein the peptide further comprises one or both of a cell-penetrating peptide sequence or a fatty acyl group, wherein the fatty acyl group is selected from a C12 to a C18 fatty acid.

Any one or more of X₉₅₈ may be A, X₉₅₉ may be A, X₉₆₀ may be A, X₉₆₁ may be A, X₉₆₂ may be A, X₉₆₃ may be A, X₉₆₄ may be A, X₉₆₆ may be A, X₉₆₇ may be A, X₉₆₈ may be A, X₉₆₉ may be A, and X₉₇₀ may be A, X₉₅₈ may be A, X₉₅₉ may be A, X₉₆₀ may be A, X₉₆₁ may be A, X₉₆₂ may be A, X₉₆₃ may be A, X₉₆₄ may be A, X₉₆₆ may be A, X₉₆₇ may be A, X₉₆₈ may be A, X₉₆₉ may be A, or X₉₇₀ may be A.

The amino acid sequence may include X₉₅₆X₉₅₇X₉₅₈SFVRNSLERRSVRM, wherein X₉₅₆ may be A or any amino acid other than P, or X₉₅₇ may be A or any amino acid other than P. The amino acid sequence may include DSSTLSKKX₉₅₆X₉₅₇X₉₅₈X₉₅₉X₉₆₀X₉₆₁X₉₆₂X₉₆₃X₉₆₄X₉₆₅X₉₆₆X₉₆₇X₉₆₈X₉₆₉X₉₇₀KRPSPPPQ

The amino acid sequence may be selected from SEQ ID NO: 1, SEQ ID NO: 2, SEQ ID NO: 3, 4 SEQ ID NO: 4, and SEQ ID NO: 5. The peptide may include a cell-penetrating peptide sequence. The cell-penetrating peptide may be selected from SEQ ID NO: 27, SEQ ID NO: 28, SEQ ID NO: 29, and SEQ ID NO: 30. The peptide may include a fatty acyl group, wherein the fatty acyl group is from a C12 to a C18 fatty acid. The peptide may include N-terminal myristoylation.

The amino acid sequence may be SEQ ID NO: 1. The amino acid sequence may be SEQ ID NO: 2. The amino acid sequence may be SEQ ID NO: 3. The amino acid sequence may be SEQ ID NO: 4. The amino acid sequence may be SEQ ID NO: 5.

### BRIEF DESCRIPTION OF THE DRAWINGS

These and other features, aspects, and advantages of the present disclosure will become better understood when the following detailed description is read with reference to the accompanying drawings, wherein:

FIGS. 1A-1C show strong synaptic stimulation facilitates formation of persistent KIBRA-PKMz complexes in late-LTP maintenance. (A) Schematic of PLA showing KIBRA-PKMz complexes detected by formation of circular DNA, which is amplified and visualized with a fluorescent probe. (B) Persistent increases of KIBRA-PKMz complexes 3 h post-tetanization in CA1 st. radiatum of mouse hippocampal slice. Above left, representative PLA images reveal complexes increase in st. radiatum (rad) and not in st. pyramidale (pyr) or lacunosum-moleculare (lac-mol), which do not receive stimulated projections. Below left, representative test responses and late-LTP, recorded in adjacent slices for 3 h. Tetanization at arrow; PLA performed at arrowheads. Right, mean=SEM. ANOVA with repeated measurement reveals significant main effect of CA1 sublayers (pyramidale, radiatum, and lacunosum-moleculare, F_{2,12}=65.35, P<0.00001, η²ₚ=0.92), as well as their interaction (F_{2,12}=20.24, P=0.0001, η²ₚ=0.77). Post hoc tests show higher PLA intensity in radiatum after LTP, compared to control (*, P<0.001), and no significant differences in pyramidale and lacunosum-moleculare (n.s., P=0.98, P=0.78, respectively, n's=4). In untetanized controls, there is no difference between pyramidale and radiatum (P=0.18), whereas lacunosum-moleculare is higher than both pyramidale and radiatum (P's<0.001). (C) Representative images of KIBRA-PKMz complexes in dendrites within st. radiatum in control and late-LTP maintenance. Scale bar in C: B, 50 μm; C, 5 μm.

FIGS. 2A-2B show both KIBRA and PKMζ probes are necessary for detection of KIBRA-PKMζ complexes in hippocampal CA1 (relevant to FIG. 1), and KIBRA and PKMζ immunofluorescence signals show no bleedthrough (relevant to FIG. 3). (A) Probes to both KIBRA and PKMzeta are required for PLA fluorescence signal. For clarity, DAPI staining of nuclei is shown. (B) KIBRA and PKMzeta fluorescence signals show no bleedthrough. For clarity, DAPI staining of nuclei is shown. CA1 strata: or, oriens; pyr, pyramidale; rad, radiatum; lac-mol, lacunosum-moleculare. Scale bar, 100 μm.

FIGS. 3A-3B show Strong synaptic stimulation facilitates persistent increases of KIBRA and PKMz in late-LTP maintenance. (A) Left, representative images of total KIBRA (green), total PKMz (red), and merged signals showing persistent increases of all three in st. radiatum 3 h after stimulation vs. control. Inserts below are from areas delineated by grey rectangles above. Right, mean±SEM. Multicomparison t-tests with Bonferroni correction (α_{adjusted}=0.017 per test) reveal KIBRA, PKMζ, and their colocalization increase 3 h after LTP induction compared to control (t₁₀=3.97, P=0.003, d=2.29; (10=5.42, P=0.0003, d=3.13; (10=3.22, P=0.009, d=1.86, respectively, n's=6). Comparing the overlap between KIBRA and PKMz signals in dendritic regions of st. radiatum by Manders coefficients M1 (KIBRA&PKMz/KIBRA) and M2 (KIBRA&PKMz/PKMz) (83) indicates PKMz is more widely distributed outside areas of colocalization than KIBRA in both control and stimulated conditions. Mean±SEM, M1 (control, 0.57±0.019; stimulated, 0.57±0.024); M2 (control, 0.50±0.025; stimulated, 0.48±0.042). ANOVA with repeated measurement reveals significant main effect of protein, F_{1,10}=64.49, P<0.0001, η²ₚ=0.87. Post hoc test shows M1 is higher than M2 in both control and stimulated conditions (P's<0.001, n's=6). (B) Representative images of colocalized KIBRA and PKMz (yellow) in puncta and shafts of dendrites in st. radiatum in control and late-LTP maintenance. Scale bar in B: A above, 50 μm; A below, 18 μm; B, 5 μm.

FIG. 4A-4B show Inhibitor of the allosteric KIBRA-binding site in PKMz blocks KIBRA-PKMz interaction. (A) Predicted structures of KIBRA (green) and PKMz (red) show ζ-stat (grey) interacting with an allosteric pocket in PKMz within the KIBRA-binding site (yellow). The KIBRA-binding site is flanked by z-specific residues, P291 and F297 (purple, amino-acid numbering based on PKMz sequence (12)). The KIBRA-binding site in PKMz is distinct from the ATP- and protein-substrate-binding sites, which are also present in the catalytic domain of PKCi/l (blue) and are inhibited by ZIP. The PKMz-binding site in KIBRA is shown in orange. (B) BiFC shows z-stat inhibits dimerization of KIBRA and PKMz. Left, above, schematic of BiFC reaction showing dimerization of KIBRA and PKMz reconstitutes fluorescence reporter split-Venus; below, suppression of BiFC by z-stat. Middle, dose-response curve shows z-stat IC₅₀ of ˜1 μM; mean±SEM, n's=4. Right, z-stat (10 μM) inhibits KIBRA interaction with PKMz, but not with PKCi/l or PKMz [PKCi/l-P291Q;F297S], in which the z-stat-binding site is changed to the amino acids in PKCi/l. Mean±SEM; n=4 sets of transfected HEK293T cultures. The two-way ANOVA reveals the main effects of drug (ζ-stat and vehicle, F_{1,16}=15.32, P<0.01, η²ₚ=0.49) and kinase (PKMz, PKCi/l, and mutated PKMz, F_{2,16}=39.42, P<0.000001, η²ₚ=0.83), and their interaction (F_{2,16}=15.23, P<0.0005, η²ₚ=0.66). Post hoc analysis shows KIBRA binds PKMz stronger than either mutated PKMz or PKCi/l (P's<0.0002), whereas binding to mutated PKMz and PKCi/l are not significantly different (P=0.06). z-stat suppresses KIBRA-PKMz coupling (*, P<0.0005), but has no effects on KIBRA-PKCi/l and KIBRA-mutated PKMz (P=0.86, P=0.76, respectively; n.s., not significant).

FIGS. 5A-5D show specificity of KIBRA-PKMζ interaction as assessed by BiFC (relevant to FIGS. 4B and 14B). Left, schematics of Venus fusion constructs used in BiFC experiments. Right, representative images of HEK293T cells 24 h after transfection. Left image column, BiFC; middle columns, immunocytochemistry for FLAG and HA; right column, merged images with nuclear stain DAPI. For clarity in the merged image, the BiFC signal is shown as blue. (A) BiFC produced by co-transfection of N-terminal-Venus FLAG-tagged KIBRA and C-terminal-Venus HA-tagged PKMζ. (B) z-stat (10 μM) decreases BIFC. (C) Minimal BiFC results from control co-transfection of the N-terminal-Venus FLAG-tagged KIBRA and the C-terminal-Venus tagged with HA. (D) Minimal BiFC following co-transfection of C-terminal-Venus HA-tagged PKMζ and N-terminal-Venus tagged with FLAG. Scale bar, 5 μm.

FIGS. 6A-6C show ζ-stat does not inhibit formation of KIBRA complexes with conventional PKCs, novel PKCs, or CaMKIIα. (A) ζ-Stat (10 μM) has no effect on BiFC produced by co-transfection of KIBRA with conventional PKCs. The two-way ANOVA reveals the main effect of conventional PKC (F_{3,24}=6.35, P<0.005, η²ₚ=0.44; PKCγ>PKCα=PKCβI=PKCβII), but no main effect of drug (ζ-stat and vehicle, F_{1,24}=0.78, P=0.39, η²ₚ=0.032) or their interaction (F_{3,24}=0.70, P=0.56, η²ₚ=0.08); n's=4. (B) z-Stat has no effect on BiFC produced by co-transfection of KIBRA with novel PKCs. The two-way ANOVA reveals the main effect of PKC (F_{3,24}=16.57, P<0.00001, η²ₚ=0.67; PKCδ>PKCε=PKCη=PKCθ), but no main effect of drug (ζ-stat and vehicle, F_{1,24}=0.59, P=0.45, η²ₚ=0.024) or their interaction (F_{3,24}=0.28, P=0.84, η²ₚ=0.034); n's=4. (C) No measurable BiFC is produced by co-transfection of KIBRA with CaMKIIα (n's=4).

FIGS. 7A-7B show Inhibitor of the KIBRA-binding site in PKMz reverses established late-LTP in wild-type mice, but not in compensated PKMz-null mice. (A) In hippocampal slices prepared from wild-type mice, z-stat (10 μM) applied to the bath 3 h post-tetanization reverses late-LTP as measured by fEPSP slope, with no effect on baseline synaptic transmission recorded in an independent synaptic pathway within the slices. Tetanization is at arrow. Above, numbered representative fEPSP traces correspond to time points noted below. Wild-type tetanized pathways: mean response 5 min before z-stat compared to 4 h after initiation of z-stat, 16=5.41, P<0.01, d=2.46, n=6; non-tetanized pathway: 5 min pre-ζ-stat vs. 4 h post-ζ-stat, (3=2.30, P=0.43; d=0.49, n=4. (B) In PKMz-knockout mice (PKMz-null), z-stat (10 μM) applied to the bath 3 h post-tetanization has no effect on late-LTP or baseline synaptic transmission. PKMz-null tetanized pathways: 5 min pre-z-stat vs. 4 h post-ζ-stat, t₃=0.79, P=0.49, d=0.23, n=4; non-tetanized pathways: 13=0.82, P=0.47, d=0.29, n=4. Wild-type compared to PKMz-null at 4 h post-z-stat: t₈=2.97, P<0.02, d=1.78.

FIGS. 8A-8C show mean±SEM for time points in physiology experiments of FIG. 7 and FIG. 9, in which ζ-stat inhibits the KIBRA-binding site in PKMz. (A) FIGS. 7, A and B; (B) FIG. 9A; (C) FIG. 9B. Statistical analyses are presented in FIG. 7 and FIG. 9 descriptions.

FIGS. 9A-9B show inhibitor of the KIBRA-binding site in PKMz reverses late-LTP, and after drug washout synaptic potentiation does not return. (A) z-stat applied 3 h post-tetanization reverses wild-type-LTP maintenance; after 4-h washout, potentiation does not return (black circles). Two-way ANOVA with repeated measurement shows effects of treatment (ζ-stat and vehicle, F_{1,7}=5.95, P<0.05, η²ₚ=0.46), time (5 min pre-HFS, 180, 360, and 500 min post-HFS, F_{3,21}=12.78, P<0.0001, η²ₚ=0.65), and interaction (F_{3,21}=9.14, P<0.0005, η²ₚ=0.57). Post hoc analysis reveals z-stat disrupts LTP (5 min response pre-tetanization vs. 5 min before z-stat, P<0.005; 5 min before vs. 5 min at end of 3-h-stat application, P<0.005); the effect persists after washout (5 min period at end of 3-h application vs. 5 min 140 min afterwards, P=0.2, n=3). LTP in vehicle is stable (grey circles, pre-tetanization vs. the 3 time points post-tetanization, P's<0.002, n=6). (B) z-stat suppresses late-LTP in one pathway, and after 1-h washout, late-LTP is induced and maintained in second pathway, indicating effective washout. ANOVA with repeated measurement reveals main effects of treatment (pathways 1 vs. 2) and time (5 min pre-tetanization vs. 150 min post-tetanization) (F_{1,8}=11.46, P<0.01, η²ₚ=0.59; F_{1,8}=80.70, P<0.0001, η²ₚ=0.91, respectively), and interaction (F_{1,8}=29.57, P<0.001, η²ₚ=0.79) Post hoc analysis reveals z-stat suppresses late-LTP (5 min pre-tetanization vs. 150 min post-tetanization with drug, P=0.08), but after 1-h washout, late-LTP is induced and maintained in second pathway (5 min pre-tetanization vs. 150 min post-tetanization without drug, P<0.0005). Post-tetanization responses with and without drug differ, P<0.0005; n's=5.

FIG. 10 shows inhibitor of the KIBRA-binding site in PKMz disrupts long-term spatial memory in wild-type mice, but not in compensated PKMz-null mice. Above left, schematic of place avoidance training apparatus shows slowly rotating arena within which is a nonrotating 60°-sector shock zone (delineated in red). Visual cues are on room walls. Above, right, conditioning protocol. Two h after a 30-min pretraining session without shock, conditioning consists of 3 trials of 30 min with 2-h intertrial intervals, producing long-term memory to avoid the shock zone. One day after conditioning, z-stat (5 nmol in 0.5 μl vehicle) or vehicle is injected in each hippocampus. Two days post-injection, memory retention is tested with shock off, measured as the time to first enter the shock zone. Below, z-stat disrupts spatial memory retention in wild-type mice, not in PKMz-null mice. Left, representative paths during pretraining, the trial at the end of training, and during retention testing with the shock off 3 days after training. Shock zone is shown red with shock on, and grey with shock off. Red circles denote where shocks occur; grey circles denote where shocks would have been received if the shock were on. Right, time to first entry measure of active place avoidance memory (mean±SEM). There is training effect (F_{2,62}=37.20, P<0.0001, η²ₚ=0.55) and interaction among effects of training, genotype, and treatment (vehicle, ζ-stat) (training X treatment: F_{2,62}=3.91, P<0.05, η²ₚ=0.11; training X genotype X treatment: F_{2,62}=3.29, P<0.05, η²ₚ=0.096). Retention in wild-type mice with ζ-stat is different from vehicle (*, P<0.005). Retention in PKM ζ-null mice with ζ-stat is not different than vehicle (n.s., not significant; P=0.70). Wild-types: n's=11; PKM ζ-nulls: vehicle, n=5, g-stat, 8.

FIGS. 11A-11B show intrahippocampal injections of z-stat cause retrograde, but once eliminated, not anterograde amnesia in wild-type mice. (A) Above, protocol shows 1 day after active place avoidance conditioning in Context A, mice are injected in hippocampus with z-stat or vehicle, and 2 days later tested for memory retention. Immediately after retention testing, the animals are conditioned in Context B, and memory for active place avoidance in Context B is tested 3 days later. Below, infusions of z-stat disrupt memory retention for the established memory (place avoidance in Context A), but do not disrupt learning or retention of a new memory (place avoidance in Context B). Left, representative paths during pretraining, the trial at the end of training, and during retention testing with the shock off 3 days after training, for the two sequential conditionings. Right, mean±SEM; * denotes significance. The time of injection is shown at arrow. There is a significant effect of training (F_{5,60}=17.96, P<0.0001, η²ₚ=0.60). Post hoc analysis reveals the z-stat-injected group is significantly different from vehicle at first retention (Context A, P<0.05), but not the second retention (Context B, P=0.95); n's=7. (B) Learning and expression of spatial memory in Context B are unaffected 2 days after z-stat disrupted the memory of Context A. Learning curve shows number of entrances for Context B (mean±SEM), Learning in Context B for mice that had previously received ζ-stat is indistinguishable from vehicle-treated mice, demonstrating ζ-stat does not suppress expression of the avoidance behavior. Two-way ANOVA shows a significant effect of training (F_{3,36}=407.1, P<0.00001, η²ₚ=0.97; Pretraining>Trial 1=Trial 2=Trial 3), and no drug effect (F_{1,12}=2.29, P=0.16, η²ₚ=0.16) or training X drug interaction (F_{3,36}=2.12, P=0.12, η²ₚ=0.15); n's=7.

FIG. 12 shows inhibitor of the KIBRA-binding site in PKMz disrupts long-term retention of fear/threat memory in wild-type mice, but not in compensated PKMz-null mice. z-stat in BLA disrupts established auditory-cued fear/threat memory. Above, protocol shows wild-type and PKM ζ-null mice undergo auditory-cued fear/threat conditioning and then retention testing 1-day post-training with the conditioned stimulus (CS) tone in a different context from that during conditioning (Test 1). The next day, mice receive z-stat (6 nmol in 0.3 μL) or vehicle into each BLA and then are tested 1-day post-injection (Test 2). The freezing responses of two phases (pre-tone and post-tone) within a retention trial are examined. Below, z-stat disrupts fear/threat memory retention in wild-type mice, but not in PKMz-null mice (mean±SEM of % time freezing). The 4-way repeated measures ANOVA (genotype x treatment x trial x phase) reveals that the main effects of treatment, trial, and phase are all significant (F_{1,39}'s>4.79, P's<0.05, η²ₚ>0.11), as are the interactions of trial x phase, trial x treatment, genotype x phase, and genotype x treatment x trial (F_{1,39}'s>5.26, P's<0.05, η²ₚ>0.12). Post hoc analysis shows z-stat disrupts the freezing response to CS in the post-injection retention test compared to the pre-injection retention test in wild-type mice (P<0.0001), but not in PKM ζ-null mice (P=0.64); wild-types: vehicle, n=11, z-stat, 14; PKMz-nulls: vehicle, n=13, z-stat, 9.

FIG. 13 shows ζ-stat in hippocampus has no effect on contextual fear/threat conditioning in wild-type mice or in PKMζ-null mice. Similar results were seen in prior experiments using ZIP (47, 48). Above, schematic of contextual fear/threat conditioning protocol. Below, mean±SEM shows no effect of drug on either mouse genotype. The 2-way ANOVA reveals no significant main effects of genotype (wild-type and PKM ζ-null, F_{1,18}=3.21, P=0.09, η²ₚ=0.15), drug (vehicle and ζ-stat, F_{1,18}=2.17, P=0.16, η²ₚ=0.11), or their interaction (F_{1,18}=0.46, P=0.50, η²ₚ=0.03). Wild-types: vehicle, n=5, D-stat, n=6; PKM ζ-nulls: vehicle, n=6, ζ-stat, n=5).

FIGS. 14A-14C show K-ZAP peptide mimicking the PKMz-binding site in KIBRA reverses late-LTP in wild-type mice, but not PKMz-null mice. (A) Predicted structures show K-ZAP mimics KIBRA PKMz-anchoring site (orange). (B) BiFC reveals K-ZAP (10 μM) inhibits PKMz-KIBRA dimerization. KIBRA-PKCi/l interactions are not measurably affected. Two-way ANOVA shows main effects of drug (K-ZAP/vehicle, F_{1,10}=14.59, P<0.005, η²ₚ=0.59), kinase (F_{1,10}=104.05, P<0.00001, η²ₚ=0.91), and interaction (F_{1,10}=19.98, P<0.005, η²ₚ=0.67). Post hoc analysis shows K-ZAP suppresses KIBRA-PKMz (P<0.0005), not KIBRA-PKCι/λ coupling (P=0.66). K-ZAP/vehicle on KIBRA-PKMz, n's=4; KIBRA-PKCi/l, n's=3. (C) K-ZAP (10 μM) reverses late-LTP (closed black circles) with no effect on untetanized pathway (open black circles) or PKMz-null (tetanized pathway, closed blue circles; untetanized pathway, open blue circles). Vehicle has no effect on wild-type-LTP (tetanized pathway, closed grey circles; untetanized, open grey circles). Tetanization at arrow. Above, numbered fEPSPs correspond to time points below. Repeated measures ANOVA shows group effect (wild-type with K-ZAP, PKMz-null with K-ZAP, and wild-type with vehicle; F_{2,9}=4.43, P<0.05, η²ₚ=0.50), time effect (5 min pre-tetanization, 5 min pre-K-ZAP, and 1 h after 3-h K-ZAP application; F_{2,18}=43.46, P<0.00001, η²ₚ=0.83), and interaction (F_{4,18}=6.32, P<0.005, η²ₚ=0.58). Post hoc analysis reveals K-ZAP disrupts established wild-type-LTP (5 min pre-tetanization vs. 5 min pre-K-ZAP, P<0.005; 5 min pre-K-ZAP vs. 1 h after 3-h application of K-ZAP, P<0.005). Wild-type-LTP remains intact in vehicle (P<0.0005, P=0.77, for equivalent time points). K-ZAP has no effect on PKMz-null-LTP (P<0.0005, P=0.21, for equivalent time points). Wild-type/K-ZAP, n=4; wild-type/vehicle, n=6; PKMz-null/K-ZAP, n=4.

FIG. 15 shows Mean±SEM for time points in physiology experiments of FIG. 14C, in which the K-ZAP peptide mimics the PKMz-binding site in KIBRA. Statistical analysis is presented in FIG. 14 description.

FIGS. 16A-16B show K-ZAP peptide mimicking PKMz-binding site in KIBRA disrupts long-term and remote spatial memory. Intrahippocampal injections of K-ZAP (5 nmol in 0.5 μl/side) disrupt maintenance of spatial memory measured (A) 3 days and, in separate experiments, (B) 30 days after conditioning. Above right insert, schematic of active place avoidance protocol; injections are 2 days before memory retention testing for 3- and 30-day experiments. (A) Above, representative paths for 3-day memory conditioning during pretraining, the trial at end of training, and during retention testing with shock off 3 days after training. The shock zone is shown in red with shock on, and grey with shock off. Red circles denote where shocks occur; grey circles denote where shocks would have been received if the shock were on. Below, mean±SEM. For 3-day memory, 2-way ANOVA (drug and genotype) with repeated measurement (training phase) shows main effect of training-phase (F_{2,44}=50.73, P<0.00001, η²ₚ=0.70) and interaction between training-phase and drug (F_{2,44}=4.57, P<0.02, η²ₚ=0.17). Post hoc analysis reveals that wild-type mice receiving K-ZAP show loss of memory retention, compared to mice receiving vehicle (P<0.01), whereas PKMζ-null mice show intact memory retention whether receiving K-ZAP or vehicle (P=0.65); wild-type mice: vehicle, n=8; K-ZAP, n=6; PKM ζ-null mice: n's=6. (B) Above, representative paths for 30-day memory. Below, 1-way ANOVA with repeated measurement shows main effect of training-phase (F_{2,28}=108.9, P<0.00001, η²ₚ=0.89) and interaction between training-phase and drug (F_{2,28}=8.63, P<0.001, η²ₚ=0.38). Post hoc analysis reveals mice receiving K-ZAP show loss of memory retention 30 days post-training, compared to mice receiving vehicle (P<0.005); vehicle, n=7; K-ZAP, n=9.

FIG. 17 shows PKMζ protein in hippocampus turns over within 30 days. PKMζ protein levels in Prkcz^{fl/fl} mice downregulate after intrahippocampal injection of an adeno-associated virus (AAV) expressing Cre-recombinase. Levels immediately after injection are set to 100% (data points shown to right of the mean symbol for clarity). PKMζ levels in AAV-Cre-injected hippocampus also decrease after 30 days compared to levels in the contralateral hippocampus injected with control AAV expressing enhanced green fluorescent protein (eGFP). Representative immunoblot shown in upper right with actin loading controls. Tests of two a priori hypotheses were conducted using Bonferroni-adjusted a levels of 0.025 per test (0.05/2). The results reveal that compared to Day 0, the amount of PKMζ is significantly suppressed after 30 days by ipsilateral injection of AAV-Cre, and not by contralateral injection of AAV-eGFP (12=26.28, P=0.001, d=15.17; t₂=0, P=1, d=0, respectively). Independent t-test confirms that the amount of PKMζ is significantly suppressed by ipsilateral injection of AAV-Cre compared to contralateral injection of AAV-eGFP on Day 30 (14=5.88, P<0.01, d=4.80; n's=3). The time to recombination of the Prkcz gene may vary among neurons after AAV-Cre injection, and the rate of turnover of PKMzeta mRNA is not known. Therefore, the rate of turnover of PKMζ protein observed in this experiment is the upper limit.

FIG. 18 shows a schematic illustration of signaling pathways for KIBRA-PKMz interaction in the induction and maintenance of late-LTP and long-term memory. In induction, high-frequency afferent synaptic stimulation activates postsynaptic N-methyl-D-aspartate receptors (NMDARs), and the resulting influx of postsynaptic Ca²⁺ initiates early-LTP and (1) increases protein synthesis (PS), which may be localized in dendrites. The enhanced synthesis results in (2) increased amounts of the postsynaptic scaffolding protein KIBRA, which interacts with AMPARs, and the persistently active kinase PKMz (FIG. 3). We hypothesize that in early-LTP, trafficking of extrasynaptic AMPARs to postsynaptic sites sequesters KIBRA at activated synapses. In late-LTP induction, KIBRA can then act as a synaptic tag to bind PKMz, shown here interacting with AMPARs as a KIBRA-PKMz dimer. In late-LTP maintenance, (3) postsynaptic KIBRA and PKMz form persistent KIBRA-PKMz complexes (FIG. 1). (4) Decoupling KIBRA from PKMz reverses late-LTP maintenance at activated synapses (FIG. 7A, and FIG. 9A, and FIG. 14C) and disrupts long-term memory maintenance for at least 4 weeks (FIG. 16B). This duration of memory is longer than the lifespans of individual KIBRA (28, 39, 40) and PKMz molecules (24, 25) (FIG. 17). Therefore, the components of the complex are likely replaced by newly synthesized molecules.

FIG. 19 shows Table 1, Plasmids and primers for cloning PKC isoforms and CaMKIIα to construct pVen2-HA-kinase fusion proteins.

### DETAILED DESCRIPTION

The autonomously active protein kinase C (PKC) isoform protein kinase Mzeta (referred to herein interchangeably as PKMz, PKMzeta, and PKMζ) (5-10) persistently potentiates synaptic transmission. PKMζ consists of the independent catalytic domain of the atypical isoform PKC. Unlike other PKCs, PKMζ lacks an autoinhibitory regulatory domain and is constitutively and thus persistently active without second messenger stimulation (11). Instead, the amount of PKMζ determines its activity (5, 11). PKMζ is selectively expressed in neurons from a dedicated PKMζ mRNA, which is transported to dendrites and under basal conditions is translationally repressed (12-15). Strong afferent synaptic stimulation derepresses the PKMζ mRNA (12). This derepression upregulates new PKMζ synthesis and increases the amount of the kinase in neurons (12, 16-19), including in dendritic spines and postsynaptic densities (20). In late-LTP maintenance recorded in hippocampal slices, the increased steady-state amount of PKMζ persists for hours in CA1 pyramidal cells (16, 17). In spatial long-term memory maintenance, the increases last for weeks in selective hippocampal neuronal circuits that were transcriptionally active during initial memory formation (19). Spatial memory formation also persistently increases PKMζ in extrahippocampal regions involved in spatial information processing such as retrosplenial cortex, but not in thalamus (19). Likewise, skilled motor learning resulting in long-term procedural memory increases PKMζ for over a month in sensorimotor cortex (21). In contrast to all other PKCs, the persistent increases in PKMζ in LTP and long-term spatial memory maintenance correlate with the extent of persistent synaptic potentiation (11, 16, 17) and memory retention (18).

ZIP, an inhibitor of PKMz's catalytic site, disrupts established late-LTP and long-term memory without affecting basal synaptic transmission (5, 7). Such an effect is disclosed in US Patent Application Publication No. US 2020/0261384 A1, which is incorporated herein by reference in its entirety for all purposes. But knockout mice lacking PKMζ (Prkcz^{-/-} mice; PKMζ-null mice) still express LTP and memory that is reversed by ZIP (22, 23). These mutant mice, however, compensate for the loss of PKMζ by the persistent activation of PKCs that show short-term increases in wild-type mice, including another atypical isoform, PKCι/λ, that is also sensitive to ZIP (17). Blocking PKMζ synthesis with shRNA or antisense-oligodeoxynucleotides that selectively suppress the translation of PKMζ mRNA, but not PKCι/λ mRNA, prevents late-LTP and long-term memory formation in wild-type animals (17, 24, 25). Thus, PKMζ synthesis is necessary for wild-type late-LTP and long-term memory, and PKMζ action is sufficient to potentiate synaptic transmission (5, 6, 17, 25).

LTP induction that increases PKMζ synthesis within a neuron (19) results in potentiation exclusively at activated synapses during LTP maintenance (17). Likewise, memory training increases PKMζ (19) as well as synaptic strength (26) in selective dendritic compartments of memory-activated hippocampal neurons for at least a month despite evidence of the kinase's rapid turnover (27, 28). Moreover, viral overexpression of PKMζ within neocortical neurons does not degrade memory, as predicted by saturating potentiation of all synapses (29, 30); instead, it enhances previously established long-term memory, presumably by strengthening a subpopulation of synapses activated during learning (31). PKMζ, however, lacks the regulatory domain by which other PKCs translocate to membrane (11). Therefore, how PKMζ action persistently targets activated synapses remains unclear, but could be through interaction with another molecule.

The inventor of the subject matter disclosed herein disclosed such subject matter in Tsokas et al., 2024, Sci Adv. 10 (26): ead10030, of which he is a co-author, the entire content of which is incorporated herein it its entirety for all purposes.

KIBRA (kidney and brain expressed adaptor protein, also known as WWC1) is a member of the WW and C2 domain-containing protein (WWC) family, and is a PKMζ-binding, postsynaptic scaffolding protein, (28, 32, 34, 35). As disclosed herein, KIBRA and PKMζ are co-expressed in neurons, synaptic stimulation facilitates persistent KIBRA-PKMζ interactions in late-LTP maintenance, and disrupting KIBRA-to-PKMζ binding disrupts molecular, cellular, neural, electrophysiological, and behavioral sequelae of previously formed long-term memory. Disrupting such binding by contacting cells with a peptide as disclosed herein may be useful therapeutically, such as administering a peptide as disclosed herein for treatment of addiction to a drug or alcohol, an anxiety disorder, neuropathic pain, or other disorder characterized by maladaptive memory-LTP-related processes or that could benefit from disruption of memory- or LTP-related processes, to a subject in need of such treatment.

A peptide as disclosed herein that prevents KIBRA-PKMζ binding may include a sequence of amino acids of KIBRA's PKMζ-binding site. Peptides having amino acid sequences corresponding to a portion or fragment of KIBRA that prevent KIBRA-PKMζ binding are disclosed in Vogt-Eisele et al., 2014, KIBRA (KIdney/BRAin protein) regulates learning and memory and stabilizes Protein kinase MG. J Neurochem. 2014, 128 (5): 686-700, which is incorporated herein in its entirety for all purposes.

Such sequences may include the amino acid sequences set out in SEQ ID NO: 1, SEQ ID NO: 2, SEQ ID NO: 3, SEQ ID NO: 4, or SEQ ID NO: 5. Such sequences may include SEQ ID NO: 6, as further described hereinbelow. A peptide having an amino acid sequence corresponding to amino acids 946-985 of KIBRA may prevent KIBRA-PKMζ binding. A peptide having an amino acid sequence corresponding to amino acids 956-975 of KIBRA may prevent KIBRA-PKMζ binding. A peptide having an amino acid sequence corresponding to amino acids 958-970 (FVRNSLERRSVRM; SEQ ID NO: 1) of KIBRA may prevent KIBRA-PKMζ binding. A peptide having an amino acid sequence corresponding to amino acids 956-970 (PPFVRNSLERRSVRM; SEQ ID NO: 2) of KIBRA may prevent KIBRA-PKMζ binding. A peptide having an amino acid sequence corresponding to amino acids 958-972 (FVRNSLERRSVRMKR; SEQ ID NO: 3) of KIBRA may prevent KIBRA-PKMζ binding. A peptide having an amino acid sequence corresponding to amino acids 948-978 (DSSTLSKKPPFVRNSLERRSVRMKRPSPPPQ; SEQ ID NO: 4) of KIBRA may prevent KIBRA-PKMζ binding. A peptide having an amino acid sequence corresponding to amino acids 958-974 (FVRNSLERRSVRMKRPS; SEQ ID NO: 4) of KIBRA may prevent KIBRA-PKMζ binding.

Such sequences may include SEQ ID NO: 6. The amino acid sequence of SEQ ID NO: 6 corresponds to the amino acid sequence of SEQ ID NO: 1, amino acids 958-970 of KIBRA, except that it may include one or more amino acid substitution compared to the amino acids of SEQ ID NO: 1. The amino acids of SEQ ID NO: 6 may be as follows: X₉₅₈X₉₅₉X₉₆₀X₉₆₁X₉₆₂X₉₆₃X₉₆₄X₉₆₅X₉₆₆X₉₆₇X₉₆₈X₉₆₉X₉₇₀, wherein X₉₅₈ through X₉₇₀ comprise acids amino acids FVRNSLERRSVRM (SEQ ID NO: 1), respectively, except that one or more of X₉₅₈ may be A or any amino acid other than F, X₉₅₉ may be A or any amino acid other than V, X₉₆₀ may be A or any amino acid other than R, X₉₆₁ may be A or any amino acid other than N, X₉₆₂ may be A or any amino acid other than S, X₉₆₃ may be A or any amino acid other than L, X₉₆₄ may be A or any amino acid other than E, X₉₆₆ may be A or any amino acid other than R, X₉₆₇ may be A or any amino acid other than S, X₉₆₈ may be A or any amino acid other than V, X₉₆₉ may be A or any amino acid other than R, and X₉₇₀ may be A or any amino acid other than M. A peptide having the sequence of amino acids set out in SEQ ID NO: 1 but including an amino acid substitution as set out in this paragraph prevents KIBRA-PKMζ binding. And such substitution may be a conservative substitution, for example. Such variations of SEQ ID NO: 6 prevent KIBRA-PKMζ binding. Vogt-Eisele et al., 2014, KIBRA (Kidney/BRAin protein) regulates learning and memory and stabilizes Protein kinase MG. J Neurochem. 2014, 128 (5): 686-700.

Any one or more of the foregoing peptide sequences may be used in a method disclosed herein, including without limitation administering to a subject a peptide, wherein the peptide prevents biding of kidney and brain expressed protein (KIBRA) to protein kinase M zeta (PKMzeta). The peptide may comprise a atty acyl group is selected from a C12 to a C18 fatty acid. The peptide may comprise a myristoyl group. The peptide may include a cell penetrating peptide sequence. The peptide may include a cell penetrating peptide sequence selected from SEQ ID NO: 27, SEQ ID NO: 28, SEQ ID NO: 29, and SEQ ID NO: 30 Administration may be intracranial. Administration may be septohippocampal administration, hippocampal administration, septal administration, amygdalar administration, cerebral cortical administration, spinal cord administration, striatal administration, or cerebellar administration. Administration may include administering to a subject in need of treatment for one or more of a drug addition, alcoholism, neuropathic pain, and an anxiety disorder.

A peptide as disclosed herein may be lipidated, by having a fatty acyl moiety bound thereto. Lipidation of a peptide may promote cellular uptake and cellular internalization of an extracellularly administered peptide, increase the half-like of a peptide administered in vivo, or both. Such a modification may be beneficial where, as disclosed herein, administration of a peptide for targeting intracellular targets such as PKMζ-to-KIBRA binding, and associated disruption of LTP and memory-related physiological process, is desired or intended. See Gao et al., 2021, Fatty acylation enhances the cellular internalization and cytosolic distribution of a cystine-knot peptide, iScience, Volume 24, Issue 11, 103220, incorporated by reference herein in its entirety for all purposes. Examples of fatty acid moieties that may be attached to a peptide for such purposes include any one or more of, without limitation, a lauroyl (C12:0), myristoyl (C14:0), palmitoyl (C16:0), or stearoyl (C18:0) fatty acyl moiety, for example. A fatty acyl moiety may be attached to a peptide by attachment to an amino group such as via formation of an amide bond, a carboxyl group such as via formation of an ester bond, a thioester bond, an aliphatic amino-group, or a mercaptan-group such as of a cysteine residue, as non-limiting examples.

Disclosed herein is a lipopeptide that prevents KIBRA-PKMζ binding, wherein the peptide has an amino acid sequence as set out in SEQ ID NO: 1, SEQ ID NO: 2, SEQ ID NO: 3, SEQ ID NO: 4, SEQ ID NO: 5, or SEQ ID NO: 6 as set out hereinabove, wherein the lipopeptide includes one or more fatty acyl group attached to the amino acid sequence. The one or more fatty acyl group attached to the peptide may include any one or more of, without limitation, a lauroyl (C12:0), myristoyl (C14:0), palmitoyl (C16:0), or stearoyl (C18:0) fatty acyl moiety, for example. A fatty acyl moiety may be attached to the peptide by attachment to an amino group such as via formation of an amide bond, a carboxyl group such as via formation of an ester bond, a thioester bond, an aliphatic amino-group, or a mercaptan-group such as of a cysteine residue, as non-limiting examples.

A peptide as disclosed herein may include as part of its amino acid sequence a cell-penetrating peptide (CPP) sequence. A CPP may be an amino acid sequence included in a peptide as disclosed herein that promotes cellular uptake and cellular internalization of an extracellularly administered peptide of which it is a part. See US Patent Application Publication US2010203611A1, US Patent Application Publication US20240317804A1, and the entireties of which are incorporated herein for all purposes. Non-limiting examples of a CPP include peptides having the amino acid sequences of SEQ ID NO: 27 (also known as Antennapedia or penetratin), SEQ ID NO: 28 (also known as TAT), SEQ ID NO: 29 (also known as transportan), SEQ ID NO: 30 (also known as polyarginine), SEQ ID NO: 31, SEQ ID NO: 32, SEQ ID NO: 33, or SEQ ID NO: 34. A peptide that prevents biding of KIBRA to PKMzeta as disclosed herein that also includes a CPP sequence is known to be capable of entering the cytoplasm of a cell following extracellular contact therewith and upon cellular entry prevents biding of KIBRA to PKMzeta. See Vogt-Eisele et al., 2014, KIBRA (KIdney/BRAin protein) regulates learning and memory and stabilizes Protein kinase Mζ. J Neurochem. 2014, 128 (5): 686-700, which is incorporated herein in its entirety for all purposes. Other examples include a cyclic CPP, a cell-penetrating peptide coupled non-covalently to a peptide that prevents KIBRA-PKMζ binding, such as Pep-1, and bacterial toxins modified for protein delivery, such as Diphtheria toxin, Anthrax toxin, and Pseudomonas exotoxin. Other types of vectors that may be used for intracellular delivery of a peptide that prevents KIBRA-PKMζ binding, in accordance with the present disclosure, include liposomes, lipid nanoparticles (LNPs), charged polymers, coordinatived polymers, boronic acid polymers, fluorinated polymers, gold nanoparticles, silica nanoparticles, virus-like particles (VLPs), exosomes, and an extracellular contractile injection system (eCIS) derived from the entomopathogenic bacterium P. asymbiotica virulence cassette (PVC). See Chan, and Tsourkas, 2024, Intracellular Protein Delivery: Approaches, Challenges, and Clinical Applications, BME Front., 5:0035, incorporated by reference herein in its entirety for all purposes.

Disclosed herein is any of the foregoing peptides that prevents KIBRA-PKMζ binding and a means for promoting cellular uptake of the peptide following extracellular contact of the peptide and a cell, wherein the means comprises any one or more of the foregoing lipidations or any one or more of the foregoing CPP sequences. Also disclosed herein is a means for preventing KIBRA-PKMζ binding, which means may be any of the foregoing peptides (including without limitation any of SEQ ID NO: 1, SEQ ID NO: 2, SEQ ID NO: 3, SEQ ID NO: 4, SEQ ID NO: 5, and any of the variants of SEQ ID NO: 6 disclosed herein), and any one or more of the foregoing lipidations or CPPs for promoting cytoplasmic uptake, of the peptide.

Learning and memory as used herein may refer to numerous molecular, cellular, neural, mental, emotional, or behavioral phenomena. In some instances, memory refers to an ability to recall particular events, emotions, facts, sensations, etc., referred to as explicit or declarative memory. In some examples, memory may involve memory for spatial relationships between places or events, and may be referred to as spatial memory. In still other examples, memory may involve memory of associations between stimuli and affective states, such as fear, hungry, affiliation, disgust (e.g., conditioned taste aversion), etc. In other examples, memory may involve a combination of any of the foregoing functions.

Memory may be related to pathological processes, such as depression or depression-related phenotypes, such as are modelled in animal models of anxiety and depression such as learned helplessness, immobilization, chronic stress administration, or forced swim paradigms. In such examples, an animal may be exposed to noxious or unpleasant stimuli and subsequent modification of behavioral responsiveness to other input that had been temporally associated with such stimuli, or of active behavioral responses to repetition of such noxious or unpleasant stimuli, measured. In such models, administration of therapeutically effective treatments for mood disorders are capable of preventing, reversing, or reducing the effects of such stimulus exposure. See Bolsoni et al., 2019, Pharmacological interventions during the process of reconsolidation of aversive memories: A systematic review, Neurobiology of Stress, 11:100194, which is incorporated by reference herein in its entirety for all purposes.

As disclosed herein, disruption of KIBRA-PKM zeta binding may block or reverse behavioral sequelae of such stimuli. For example, exposing rodents to chronic stress is known to have anxiogenic effects in several behavioral paradigms such as the open field test and the elevated plus maze, and to induce a depression-like phenotype in animal models such as causing increased immobility in the forced swim test. Exposure to chronic stress also increases expression of PKM zeta in the brain, and inhibition of PKC zeta/PKM zeta activity reduces these behavioral effects of chronic stress, indicating that it functions like an anxiolytic compound or antidepressant compound. In accordance with the present disclosure, a peptide that inhibits KIBRA-PKMz binding may be administered to subjects, such as animals or humans, subjected to various noxious or unpleasant stimuli to reverse the affective and behavioral consequence of such stimulus exposure. A peptide that prevents KIBRA-PKMζ binding to, for example, the amygdala, hippocampus, or prefrontal cortex may reduce anxiety or have antianxiety effects, including possibly as a treatment for anxiety-related behaviors such as post-traumatic stress disorder, or reduce depression or have antidepressant action, following administration to a subject in need of such treatment. See Ji et al., 2014, Intra-hippocampal administration of ZIP alleviates depressive and anxiety-like responses in an animal model of posttraumatic stress disorder. Behav Brain Funct., 10:28 and Liu, 2022, Involvement of PKMζ in Stress Response and Depression, Front Cell Neurosci., 16:907767, which references are hereby incorporated herein by reference in their entireties for all purposes.

As further disclosed herein, mood disorders believed to result from persistent memory functions related to neural processes underlying negative affect or recollection of or perseverative cognition of painful, frightening, or unpleasant stimuli may result from KIBRA-PKMz binding, reinforcing activity of neural systems responsible for such persistent memory functions. As disclosed herein, a peptide that prevents KIBRA-PKMzeta binding may be administered as a treatment for such mood disorders. For example, as disclosed herein, a peptide that prevents KIBRA-PKMzeta binding may be administered as a treatment for post-traumatic stress disorder, depression, anxiety, or phobia. In some examples, such disorders are or may have been caused by prior experiences, and therefore reducing binding of KIBRA to PKM zeta may function to reduce or eliminate pathological processes caused by such experiences and causally related to such mood disorders or symptoms thereof. For example, a person may have been exposed to traumatic experiences and developed an affective disorder requiring treatment, such as a treatment to alleviate or reduce memory-related mechanisms triggered by such experiences resulting in the affective or mood disorder. A peptide that prevents KIBRA-PKMzeta binding may be administered to such individuals to effect such treatment.

A peptide that prevents KIBRA-PKMzeta binding may be administered as an adjunctive therapy along with treatment with a different therapy such as a pharmacological therapy, behavioral therapy, psychotherapy, shock therapy, or other treatment for affective disorders. A peptide that prevents KIBRA-PKMzeta binding may be administered to individuals undergoing cognitive, behavioral, or psychoanalytical therapy, whether in addition to other pharmacological treatment or not, where such treatment is performed for the purpose of extinction of memories with pathological influence. For example, treatment for emotional disorders such as anxiety disorders, post-traumatic stress disorder, or phobias, fears, or other maladies typified by fixation on aversive, anxiety-provoking, or stressful memories may involve modification of neural processes underlying memory. During treatment, it may be beneficial to modify the emotional significance of valence a subject associates with such a memory. Evocation of aversive memories involves a process referred to as reconsolidation, during which aversive memories transiently become labile and subject to modification. Pharmacological interventions that promote, enhance, trigger, prolong, or otherwise modify a reconsolidation window during which memories become labile for reconsolidation are considered useful as treatment or adjunctive treatment for mood disorders. Administering a pharmacological intervention that is conducive to rendering established memories transiently labile is an option for enhancing the efficacy of treatment premised on reducing pathological impacts of aversive memories on affect and cognition. See Bolsoni et al, 2019, Pharmacological interventions during the process of reconsolidation of aversive memories: A systematic review, Neurobiology of Stress, 11:1000194, the entirety of which is incorporated herein by reference for all purposes. A peptide that prevents KIBRA-PKMzeta binding as disclosed herein may promote transient lability of memory during a reconsolidation window to enhance effectiveness of treatment for anxiety disorders such a post-traumatic stress disorder and other disorders and pathological conditions resulting from aversive memories.

Exposure to positively motivating stimuli may affect subsequent responsiveness to such stimuli or other input spatially or temporally associated therewith. For example, consumption of drugs of abuse such as cocaine, opiates, amphetamines, marijuana or cannabinoids, nicotine, or other stimulants, narcotics, anesthetics, anxiolytics, or to alcohol, or other addictive substances may alter neural function resulting in pathological behaviors directed towards continued consumption of such stimuli. Memory-related mechanisms are known to be engaged in the behavioral and affective changes that follow from exposure to drugs of abuse. In animal models, inhibition of PKC zeta/PKM zeta is known to impair behavioral sequelae of exposure to drugs of abuse, in models considered animal models of addiction. In such models, treatments that prevent, reduce, or reverse the affective or behavioral effects of exposure to drugs of abuse may be effective as treatments for drug addiction or alcohol. For example, conditioned placed preference models, self-administration models, and locomotor sensitization models are examples of animal models of drug addiction. For example, exposure to morphine increase PKM zeta expression in brain regions known to be important in development of drug addiction, and inhibiting PKC zeta/PKM zeta activity prevents behavioral modification caused by exposure to morphine such as development of a conditioned place preference. Administering a peptide that prevents KIBRA-PKMz binding, as disclosed herein, may prevent, reverse, or reduce the behavioral or affective sequelae of drug or alcohol exposure in such models. As further disclosed herein, administering a peptide that prevents KIBRA-PKMz binding to humans may be used for prevention of drug or alcohol craving or drug or alcohol seeking behavior, or otherwise as a treatment for drug addiction or alcoholism. For example, a peptide that prevents KIBRA-PKMz binding may be administered to drug-addicted individuals, in need of a treatment to prevent drug-seeking, drug-taking, drug-craving, or relapse from abstinence. A peptide that prevents KIBRA-PKMz binding may be administered on its own, or may be administered as an adjunct to other therapy for addiction or alcoholism. In some examples, a peptide that prevents KIBRA-PKMz binding may be administered to individuals undergoing cognitive, behavioral, or psychoanalytical therapy, whether in addition to other pharmacological treatment or not, where such treatment is performed for the purpose of treating drug addiction or alcoholism. See Li et al., 2011, Inhibition of PKMζ in Nucleus Accumbens Core Abolishes Long-Term Drug Reward Memory, Journal of Neuroscience 31 (14) 5436-5446; Crespo et al. (2012) Activation of PKCzeta and PKMzeta in the Nucleus Accumbens Core Is Necessary for the Retrieval, Consolidation and Reconsolidation of Drug Memory. PLOS ONE 7 (2): e30502; He et al., 2011, PKMζ maintains drug reward and aversion memory in the basolateral amygdala and extinction memory in the infralimbic cortex, Neuropsychopharmacology, 36 (10): 1972-81; Santerre et al., 2014, Ethanol dose-dependently elicits opposing regulatory effects on hippocampal AMPA receptor GluA2 subunits through a zeta inhibitory peptide-sensitive kinase in adolescent and adult Sprague-Dawley rats, Neuroscience, 280:50-9; Vélez-Hernández et al., 2013, Inhibition of Protein kinase Mzeta (PKMζ) in the mesolimbic system alters cocaine sensitization in rats, J Drug Alcohol Res, 2:235669; Lee et al 2014, Deletion of Prkcz increases intermittent ethanol consumption in mice, Alcohol Clin Exp Res, 38 (1): 170-8; Braren et al., Methamphetamine-induced short-term increase and long-term decrease in spatial working memory affects protein Kinase M zeta (PKMζ), dopamine, and glutamate receptors, Front Behav Neurosci., 8:438; and Xue et al., 2012, A memory retrieval-extinction procedure to prevent drug craving and relapse, Science, 336 (6078): 241-5; the entireties of which references are hereby incorporated by reference herein for all purposes.

Stimuli or experience may affect neural function leading to aberrant, persistent sensation of pain, or hypersensitivity to previously mildly painful stimuli, or an ability of previously non-painful stimuli to cause pain. Referred to generally as neuropathic pain, such functions are known to involve activity of PKC zeta/PKM zeta for their maintenance. Examples may include phantom limb pain, nerve damage, nerve trauma, neuropathy (e.g., diabetic neuropathy), cancer pain, or other known or undiagnosed causes of paid related to aberrant neural function. For example, damage to somatosensory peripheral nerves can modify central nervous system processes such that pain may be perceived in the absence of application of pain-inducing stimuli, and/or perceptual or behavioral responses to noxious or painful stimuli may become enhanced or exaggerated (e.g., previously mildly noxious or painful stimuli may come to elicit a higher degree of pain). Neural mechanisms related to memory, such as the formation or maintenance of LTP, are known to be involved in neuropathic pain. Activity of PKC zeta/PKM zeta in the nervous system is known to be involved in regulation of neural processing attendant to development of neuropathic pain in response to different stimuli and experiences, and inhibition of PKC zeta/PKM zeta diminishes indices of neuropathic pain in animal models related to such phenomena. For example, in animal models, such as the mechanical allodynia test, inhibition of PKC zeta/PKM zeta activity increases the threshold for tactile stimuli to induce a withdrawal response following peripheral nerve injury, indicating inhibition of neuropathic pain processes. As disclosed herein, a peptide that prevents KIBRA-PKMz binding may be administered as a treatment for neuropathic pain, such as to reduce human a patient's or patients' persistent sensation or perception of pain caused by or consequent to pathophysiological processes or physiological damage or disruption to normal physiological processes related to perception or sensation of pain. See Marchand et al., 2011, Specific involvement of atypical PKCζ/PKMζ in spinal persistent nociceptive processing following peripheral inflammation in rat, Mol Pain, 7:86; Nasir et al., 2016, Consistent sex-dependent effects of PKMζ gene ablation and pharmacological inhibition on the maintenance of referred pain, Mol Pain., 12:1744806916675347; Han et al., 2015, Plasticity-Related PKMζ Signaling in the Insular Cortex Is Involved in the Modulation of Neuropathic Pain after Nerve Injury, Neural Plast; 2015:601767; King et al., Contribution of PKMζ-dependent and independent amplification to components of experimental neuropathic pain, Pain, 153 (6): 1263-1273; Tang et al., 2016, Zeta Inhibitory Peptide as a Novel Therapy to Control Chronic Visceral Hypersensitivity in a Rat Model, PLOS One, 11 (10): e0163324; Asiedu et al., 2011, Spinal protein kinase Mζ underlies the maintenance mechanism of persistent nociceptive sensitization, J Neurosci.; 31 (18): 6646-53; Chen, Involvement of protein kinase (in the maintenance of hippocampal long-term potentiation in rats with chronic visceral hypersensitivity, J Neurophysiol, 113 (9): 3047-55; Melemedjian et al, 2013, BDNF regulates atypical PKC at spinal synapses to initiate and maintain a centralized chronic pain state, Mol Pain, 9:12; and Laferrière et al., 2011, PKMζ is essential for spinal plasticity underlying the maintenance of persistent pain, Mol Pain, 7:99, the entireties of which references are hereby incorporate herein by reference herein for all purposes.

Percent (%) sequence identity refers to the percentage of amino acid (or nucleic acid) residues of a sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

A peptide that prevents KIBRA-PKMζ binding may include one or more amino acid substitution, including a conservative amino acid substitution. Thus, it may include an amino acid sequence that has less than 100% sequence identity with SEQ ID NO: 1, SEQ ID NO: 2, SEQ ID NO: 3, SEQ ID NO: 4, or SEQ ID NO: 5. It may include one amino acid substitution, such as a conservative amino acid substitution, compared to a sequence as set out in SEQ ID NO: 1, SEQ ID NO: 2, SEQ ID NO: 3, SEQ ID NO: 4, or SEQ ID NO: 5. It may have 1, 2, 3, 4, 5, 6, 7, 8, 9, 10, 11, 12, 13, 14, 15, 16, 7, 18, 19, 20, 21, 22, 23, 24, 25, 26, 27, 28, 29, 30, or 31 amino acid substitutions, including 1, 2, 3, 4, 5, 6, 7, 8, 9, 10, 11, 12, 13, 14, 15, 16, 7, 18, 19, 20, 21, 22, 23, 24, 25, 26, 27, 28, 29, 30, or 31 conservative amino acid substitutions, provided the resulting peptide prevents KIBRA-PKMζ binding.

The term “amino acid” or “any amino acid” as used here refers to any and all amino acids (i.e. organic molecules including an amino group and a carboxyl group, connected by a central carbon atom and including a side chain), including naturally occurring amino acids (e.g., α-amino acids, wherein the side chain is attached directly to the central carbon), unnatural amino acids, modified amino acids, and non-natural amino acids. It includes both D- and L-amino acids. Natural amino acids include those found in nature, such as, e.g., 23 aforementioned amino acids that combine into peptide chains to form the building-blocks of a vast array of proteins. These are primarily L stereoisomers, although a few D-amino acids occur in bacterial envelopes and some antibiotics. “Unnatural” or “non-natural” amino acids are non-proteinogenic amino acids (i.e., those not naturally encoded or found in the genetic code) that either occur naturally or are chemically synthesized. Over 140 unnatural amino acids are known and thousands of more combinations are possible. Examples of “unnatural” amino acids include β-amino acids (β3 and β2), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety, such as attached directly to the carboxyl or amino group or to the side chain, not naturally present on the amino acid and are included as examples where an amino acid is referred to herein.

One or more amino acid in an immunogenic polypeptide as disclosed herein may be an R-amino acid or an L-amino acid. One or more amino acid in an immunogenic polypeptide as disclosed herein may be a standard amino acid (i.e., selected from Alanine, Arginine, Asparagine, Aspartic Acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, Valine, Selenocysteine, N-formylmethionine, and Pyrrolysine).

An amino acid of one type of class may be substituted by another amino acid in the same class, or having similar chemical or physical properties, as would be understood by skilled persons, in what is referred to as a conservative substitution. A conservative substitution is one in which an amino acid is substituted for another amino acid that has similar properties, such that one skilled in the art of peptide chemistry would expect the secondary structure and hydropathic nature of the polypeptide to be substantially unchanged. In general, a substitution of one amino acid within the following groups for another amino acid within the following groups represents a conservative substitution: (1) Aliphatic amino acids Glycine (Gly, G), Alanine (Ala, A), Valine (Val, V), Leucine (Leu, L), Isoleucine (Ile, I); (2) hydroxyl or sulfur/selenium-containing Serine (Ser, S), Cysteine (Cys, C), Selenocysteine (Sec, U), Threonine (Thr, T), Methionine (Met, M); Cyclic Proline (Pro, P); Aromatic Phenylalanine (Phe, F), Tyrosine (Tyr, Y), Tryptophan (Trp, W); Basic Histidine (His, H), Lysine (Lys, K), Arginine (Arg, R); Acidic and their amides Aspartate (Asp, D), Glutamate (Glu, E), Asparagine (Asn, N), Glutamine (Gln, Q).

As used herein, the terms “treatment” or “treating,” or “palliating” or “ameliorating” refer to an approach for obtaining beneficial or desired results including but not limited to therapeutic benefit and/or a prophylactic benefit. A peptide that prevents KIBRA-PKMζ binding as disclosed herein may be administered to reduce severity an anxiety disorder, or a depressive disorder, or post-traumatic stress disorder, or a disorder of neuropathic pain such as phantom limb pain or other neuropathic pain condition such as secondary to another illness or condition such as cancer, or to treat a drug addition or alcoholism, following administration of the peptide to a subject.

Administering may include administering orally, intramuscularly, subcutaneously, intraperitoneally, intrathecally, or intracranially. The organism to which the peptide preventing KIBRA-PKMζ bin ling is administered may include a rodent or a primate. The peptide may interfere with long-term memory retrieval or maintenance of long-term memory, and LTP-related processes. Long-term memory retrieval may include spatial memory, emotional memory, addiction, neuropathic pain, visual recognition memory, declarative memory, or episodic memory. The peptide may be administered in a route or formulation suitable for contacting neural tissue with the peptide, such as intracerebral injection, intracerebroventricular injection, intrathecal injection, or other route of administration. Such neural tissue contacted by a peptide following administration, including without limitation neural tissue into which the peptide may be injected intracerebrally, may be cortical tissue, septal tissue, hippocampal tissue, or septohippocampal tissue, amygdalar tissue, striatal tissue, spinal cord tissue, striatal tissue, or cerebellar tissue. The peptide may have been admixed with a pharmaceutically acceptable excipient or carrier. Formulations for administration to a subject include, without limitation, include those suitable for oral, parenteral (including subcutaneous, intradermal, intramuscular, intravenous and intraarticular), rectal and topical (including dermal, buccal, sublingual and intraocular) administration, or any and all other routes or methods of administration as further disclosed herein. The most suitable route may depend upon the condition and disorder of a recipient or intended purpose of the administration. A formulation may conveniently be presented in unit dosage form and may be prepared by any of the methods well known in the art of pharmacy. Methods may include a step of bringing into association a peptide that prevents KIBRA-PKMζ binding or a pharmaceutically acceptable salt thereof (“active ingredient”) with a carrier which constitutes one or more accessory ingredients. In general, formulations may be prepared by uniformly and intimately bringing into association an active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of an active ingredient, as a powder or granules; as a solution or a suspension in an aqueous liquid or a non-aqueous liquid; or as an oil-in-water liquid emulsion or a water-in-oil liquid emulsion. A peptide that prevents KIBRA-PKMζ binding may also be presented as a bolus, electuary or paste. For oral or other administration, a peptide that prevents KIBRA-PKMζ binding may be suspended in a solution, or dissolved in a solvent, such as alcohol, DMSO, water, saline, or other solvent, which may be further diluted or dissolved in another solution or solvent, and may or may contain a carrier or other excipient in some examples.

In certain embodiments, a peptide that prevents KIBRA-PKMζ binding may be incorporated with excipients and used in the form of ingestible tablets, buccal tablets, troches, capsules, elixirs, suspensions, syrups, wafers, and the like. Tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

A tablet may be made by compression or molding, optionally with one or more accessory ingredients. Compressed tablets may be prepared by compressing in a suitable machine an active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. Tablets may optionally be coated or scored and may be formulated so as to provide sustained, delayed or controlled release of an active ingredient therein.

Formulations for parenteral, or intracerebral, or intrathecal, or intracerebroventricular, or oral, or other administration include aqueous and non-aqueous sterile injection solutions which may contain anti-oxidants, buffers, bacteriostats and solutes which render a formulation isotonic with the blood of the intended recipient. Formulations for parenteral or other administration also may include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of a peptide that prevents KIBRA-PKMζ binding to polymer and the nature of the particular polymer employed, the rate of release of a peptide that prevents KIBRA-PKMζ binding can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

A formulation of a peptide that prevents KIBRA-PKMζ binding may include different types of carriers depending on whether it is to be administered in solid, liquid or aerosol form, and whether it needs to be sterile for such routes of administration as injection. It may be administered intravenously, intradermally, transdermally, intratbecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990.

The term “pharmaceutically acceptable salt” refers to salts prepared from pharmaceutically acceptable non-toxic acids or bases including inorganic acids and bases and organic acids and bases. When a composition including a peptide that prevents KIBRA-PKMζ binding is basic, salts may be prepared from pharmaceutically acceptable non-toxic acids including inorganic and organic acids. Suitable pharmaceutically acceptable acid addition salts for the compounds of the as disclosed herein include acetic, adipic, alginic, ascorbic, aspartic, benzenesulfonic (besylate), benzoic, betulinic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, ursolic and the like. When the compounds contain an acidic side chain, suitable pharmaceutically acceptable base addition salts for the compounds as disclosed herein include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

A peptide that prevents KIBRA-PKMζ binding may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

As used herein, the term “physiologically functional derivative” refers to any pharmaceutically acceptable derivative of a compound as disclosed herein that, upon administration to a mammal, is capable of providing (directly or indirectly) a compound as disclosed herein or an active metabolite thereof. Such derivatives, for example, esters and amides, will be clear to those skilled in the art, without undue experimentation. Reference may be made to the teaching of Burger's Medicinal Chemistry And Drug Discovery, 5 th Edition, Vol 1: Principles and Practice.

As used herein, the term “effective amount” means an amount of pharmaceutical agent including a peptide that prevents KIBRA-PKMζ binding that may elicit a biological or medical response of a cell, tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a peptide that prevents KIBRA-PKMζ binding, as well as salts, solvates, and physiological functional derivatives thereof, may be administered as the raw chemical. Additionally, the active ingredient may be presented as a pharmaceutical composition.

Pharmaceutical compositions as disclosed herein may include an effective amount of a peptide that prevents KIBRA-PKMζ binding and optionally one or more additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains a peptide that prevents KIBRA-PKMζ binding and optionally one or more additional active ingredient will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

Further in accordance with the present disclosure, a composition suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods disclosed herein is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, a peptide that prevents KIBRA-PKMζ binding may be combined with a carrier in any convenient and practical manner, i.e., by solution, suspension, emulsification, admixture, encapsulation, absorption and the like. Such procedures are routine for those skilled in the art.

As disclosed herein, the composition may be combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

Subject matter disclosed herein may concern the use of pharmaceutical lipid vehicle compositions that include a peptide that prevents KIBRA-PKMζ binding and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man) However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

One of ordinary skill in the art would be familiar with the range of techniques that can be employed for dispersing a composition in a lipid vehicle. For example, a peptide that prevents KIBRA-PKMζ binding may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to a subject (e.g., an animal or human patient) can be determined by physical and physiological factors such as body weight, severity of condition, the type of disease being treated, previous or concurrent therapeutic interventions, idiopathy of the patient and on the route of administration, and purpose of treatment. Depending upon the dosage and the route of administration, the number of administrations of a preferred dosage and/or an effective amount may vary according to the response of the subject or purpose of treatment. The practitioner responsible for administration will, in any event, determine the concentration of active ingredient(s) in a composition and appropriate dose(s) for the individual subject.

In other non-limiting examples, a dose may also comprise from about 1 microgram/kg body weight, about 5 microgram/kg body weight, about 10 microgram/kg body weight, about 50 microgram/kg/body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram/kg body weight to about 500 milligram/kg body weight, etc., can be administered, based on the numbers described above.

Dosing can be modified or chosen based on factors including purpose of treatment, severity of symptoms, or an individual subject's body mass. A daily dose may be administered once per day, or distributed over 2, 3, 4, 5, 6, 7, 8, or more administrations per day. A daily dose may be between 10 mg and 20 g per day. A daily dose may be less than 10 mg, for example 5 mg or 1 mg per day, or in a range of between 1-5 mg or between 5-10 mg. A daily dose may be between 10 mg and 50 mg, or between 50 mg and 100 mg, or between 100 mg and 150 mg, or between 150 mg and 200 mg, or between 200 mg and 250 mg, or between 250 mg and 300 mg, or between 300 mg and 350 mg or between 350 m and 400 mg or between 400 mg and 450 mg or between 450 mg and 500 mg. A daily dose may be between 500 mg and 600 mg, or between 600 mg and 700 mg, or between 700 mg and 800 mg, or between 900 mg and 1 g, or between 1 g and 1500 mg, or between 1500 mg and 2 g, or between 2 g and 2500 mg, or between 2500 mg and 3 g, or between 3 g and 3500 mg, or between 3500 mg and 4 g, or between 4 g and 4500 mg, or between 4500 mg and 5 g. A daily dose may be between 5 g and 6 g, or between 6 g and 7 g, or between 7 g and 8 g, or between 8 g and 9 g, or between 9 g and 10 g, or between 10 g and 11 g, or between 11 g and 12 g, or between 12 and 13 g, or between 13 g and 14 g, or between 14 g and 15 g, or between 15 g and 16 g, or between 16 g and 17 g, or between 17 g and 18 g, or between 18 g and 19 g, or between 19 g and 20 g. All subranges within and between any of these ranges are also included within the present disclosure.

In some embodiments, a peptide that prevents KIBRA-PKMζ binding may be formulated to be administered via an alimentary route. Alimentary routes include all possible routes of administration in which the composition is in direct contact with the alimentary tract. Specifically, a peptide that prevents KIBRA-PKMζ binding may be administered orally, buccally, rectally, or sublingually. As such, a peptide that prevents KIBRA-PKMζ binding may be formulated with an inert diluent or with an assimilable edible carrier, or may be enclosed in hard- or soft-shell gelatin capsule, or may be compressed into tablets, or may be incorporated directly with the food of the diet.

For oral administration a peptide that prevents KIBRA-PKMζ binding may alternatively be incorporated with one or more excipients in the form of a mouthwash, dentifrice, buccal tablet, oral spray, or sublingual orally administered formulation. For example, a mouthwash may be prepared incorporating a peptide that prevents KIBRA-PKMζ binding in the required amount in an appropriate solvent, such as a sodium borate solution (Dobell's Solution). Alternatively, a peptide that prevents KIBRA-PKMζ binding may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, a peptide that prevents KIBRA-PKMζ binding may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In further embodiments, the a peptide that prevents KIBRA-PKMζ binding may be administered via a parenteral route. As used herein, the term “parenteral” includes routes that bypass the alimentary tract. Specifically, a peptide that prevents KIBRA-PKMζ binding may be administered for example, but not limited to intravenously, intradermally, intramuscularly, intraarterially, intrathecally, subcutaneous, or intraperitoneally.

Solutions of a peptide that prevents KIBRA-PKMζ binding as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form may be sterile and fluid to the extent that easy injectability exists. A carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution may be suitably buffered if necessary and a liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety and purity standards as required by FDA Office of Biologics standards.

Sterile injectable solutions may be prepared by incorporating a peptide that prevents KIBRA-PKMζ binding in the required amount in the appropriate solvent with various of the other ingredients enumerated above, as required, followed by filtered sterilization. Generally, dispersions may be prepared by incorporating various sterilized active ingredients into a sterile vehicle which contains the basic dispersion medium and the required other ingredients from those enumerated above. In the case of sterile powders for the preparation of sterile injectable solutions, preferred methods of preparation include vacuum-drying and freeze-drying techniques which yield a powder of the active ingredient plus any additional desired ingredient from a previously sterile-filtered solution thereof. A powdered composition may be combined with a liquid carrier such as, e.g., water or a saline solution, with or without a stabilizing agent.

In other embodiments a peptide that prevents KIBRA-PKMζ binding may be formulated for administration via various miscellaneous routes, for example, topical (i.e., transdermal) administration, mucosal administration (intranasal, vaginal, etc.) and/or inhalation.

Pharmaceutical compositions for topical administration may include a peptide that prevents KIBRA-PKMζ binding formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described. Likewise, the delivery of drugs using intranasal microparticle resins and lysophosphatidyl-glycerol compounds are also well-known in the pharmaceutical arts. Likewise, transmucosal drug delivery in the form of a polytetrafluoroetheylene support matrix may be adopted for use in accordance with the present disclosure.

The term aerosol refers to a colloidal system of finely divided solid of liquid particles dispersed in a liquefied or pressurized gas propellant. An aerosol for inhalation may consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### EXAMPLES

The following examples are intended to illustrate particular embodiments of the present disclosure, but are by no means intended to limit the scope thereof.

### Materials and Methods

### Experimental Design

### Reagents

Unless otherwise stated, reagents were from MilliporeSigma. ζ-stat (1-naphthol-3,6,8-trisulphonic acid, NSC 37044, referred to herein interchangeably as z-stat, ζ-stat, or zeta-stat) was obtained from Drug Synthesis and Chemistry Branch, Developmental Therapeutics Program, Division of Cancer Treatment and Diagnosis, National Cancer Institute. Upon arrival the drug was dissolved in phosphate-buffered saline (PBS [pH 7.4]), aliquoted at 10 mM stock solution, and stored at −20° C. K-ZAP (myr-N-FVRNSLERRSVRMKRPS-C) was custom-synthesized by AnaSpec (Fremont, CA) and stored in PBS [pH 7.4] at −20° C.

### Animals and Cell-Lines

This study was performed in strict accordance with the recommendations in the Guide for the Care and Use of Laboratory Animals of the National Institutes of Health. All animals were handled according to approved institutional animal care and use committee (IACUC) protocols (#11-10274, #15-10467 of the State University of New York (SUNY) Downstate Health Sciences University or #2000-4512 of McGill University). The protocols were approved by the Institutional Animal Care and Use Committee of SUNY Downstate Health Sciences University (Animal Welfare Assurance Number: D16-00167) and McGill University (Animal Welfare Assurance Number: F16-00005). All efforts were made to minimize animal suffering and to reduce the number of animals used. C57/B6 mice and PKMζ-null mice on a C57/B6 background at SUNY Downstate Health Sciences University were genotyped as previously described (17). PKMζ-null mice at McGill University were a generous gift from Wayne Sossin, Montreal Neurological Institute, McGill University. Prkcz^{fl/fl} mice were a generous gift from Sourav Ghosh, Yale University. Male mice were examined in this study, and KIBRA-PKMζ interaction in LTP and memory maintenance in both sexes will be compared in a future study. HEK293T cell-line was obtained from the American Type Culture Collection (ATCC).

### Hippocampal Slice Recording and Stimulation

Acute mouse hippocampal slices (450 μm) were prepared as previously described (17, 73). Hippocampi from 2-6-month-old male C57/B6 or PKM ζ-null mice as previously described (17) were dissected, bathed in ice-cold dissection buffer, and sliced with a McIlwain tissue slicer in a cold room (4° C.). The dissection buffer contained (in mM): 125 NaCl, 2.5 KCl, 1.25 NaH₂PO₄, 26 NaHCO₃, 11 glucose, 10 MgCl₂, and 0.5 CaCl₂), and was bubbled with 95% O₂/5% CO₂ to maintain pH at 7.4. After dissection the slices were immediately transferred into an Oslo-type interface recording chamber (31.5±1° C.) (73). The recording superfusate consisted of (in mM): 118 NaCl, 3.5 KCl, 2.5 CaCl₂), 1.3 MgSO₄, 1.25 NaH₂PO₄, 24 NaHCO₃, and 15 glucose, bubbled with 95% O₂/5% CO₂, with a flow rate of 0.5 ml/min. In a subset of experiments a custom-made recirculation system employing piezoelectric pumps was used for recycling the superfusate (Bartels Mikrotechnik GmbH, Dortmund, Germany) (73).

Field EPSPs were recorded with a glass extracellular recording electrode (2-5 M (2) placed in the CA1 st. radiatum, and concentric bipolar stimulating electrodes (CBBRE75 and 30200; FHC, Bowdoin, ME) were placed on either side within CA3 or CA1. Test stimulation rate was once every 30 sec, alternating every 15 sec between stimulating electrodes. Based upon a pre-established exclusion criterion, a slice was not used if fEPSP spike threshold was <2 mV on initial input-output analysis. Pathway independence was confirmed by the absence of paired-pulse facilitation between the two pathways. A single stimulating electrode was used for PLA/immunocytochemistry with a test stimulation rate of once every 30 sec. The high-frequency stimulation, optimized to produce a relatively rapid onset of protein synthesis-dependent late-LTP (74), consisted of two 100 Hz-1 s tetanic trains, at 25% of spike threshold, spaced 20 sec apart. The maximum slope of the rise of the fEPSP was analyzed on a PC using the WinLTP data acquisition program (75).

### Proximity Ligation Assay (PLA)

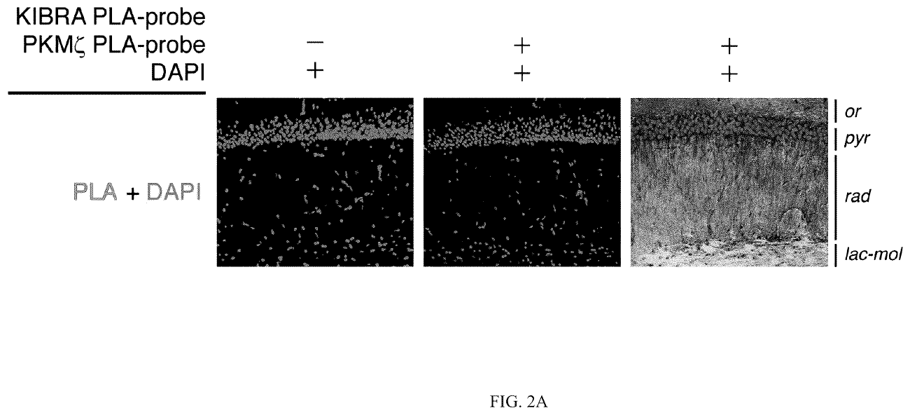

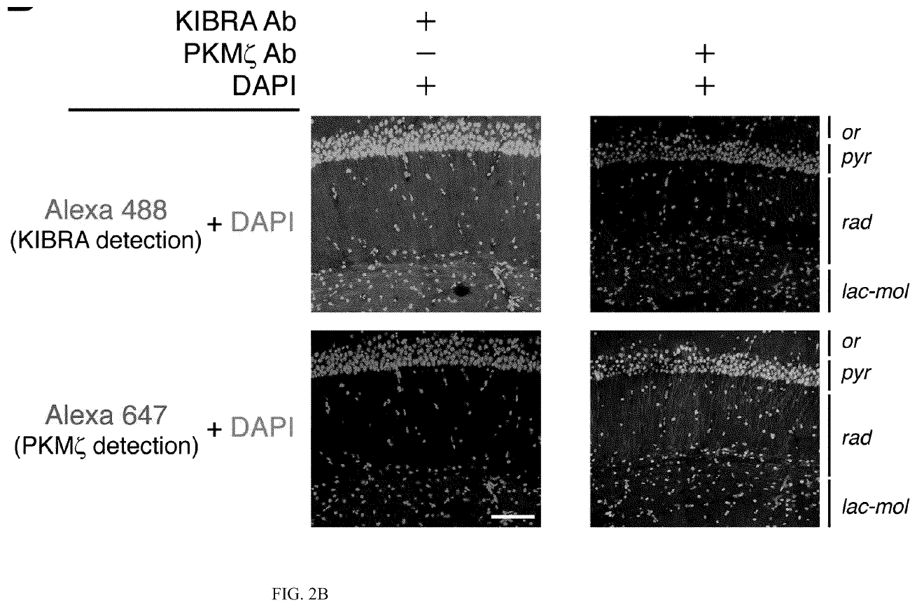

Methods used were as described in the Sigma Duolink PLA Probemaker Guide (MilliporeSigma st. Louis, MO). Because PLA is highly sensitive, standard immunocytochemical blocking methods used to detect independent fluorescent signals from two primary antisera of the same species (FIG. 2B) may not be sufficient; therefore, anti-PKMζ-PLUS and anti-KIBRA-MINUS probes were generated by directly conjugating the individual rabbit primary antibodies (C2 (12, 19) and ab216508, Abcam, Waltham, MA) with PLA oligonucleotides (PLUS and MINUS, respectively). Briefly, following purification with Microcon Centrifugal Filters (Millipore, Burlington, MA), the carrier- and preservative-free anti-PKMζ and anti-KIBRA primary antibodies (1 mg/mL in PBS) were incubated with lyophilized nucleotides (PLUS or MINUS, respectively) at 20° C. overnight. The reaction was terminated by incubation with the Stop Reagent for 30 min at 20° C., and an equal total volume of Storage Solution was added.

Hippocampal slices were fixed by immersion in ice-cold 4% paraformaldehyde in 0.1 M phosphate buffer (PB [pH 7.4]) immediately after recording, and post-fixed for 48 h. Slices were then washed with PBS [pH 7.4] and cut into 20 μM sections using a Leica VT 1200S vibratome. Free-floating sections were permeabilized in 96-well plates with PBS-TX100 for 6×10 min at 20° C., and blocked with Duolink Blocking Solution (Sigma-Aldrich DUO82007-8 ml) for 1 h at 37° C. in a pre-heated humidity chamber. The sections were then incubated overnight at 4° C. with anti-PKMζ-PLUS Probe (1:400) and anti-KIBRA-MINUS Probe (1:400) mixed in Duolink Probe Diluent. After 6×10 min washes with Duolink In Situ Buffer A, the sections were incubated in a pre-heated humidity chamber for 30 min at 37° C. with Ligation Buffer (5× Duolink Ligation buffer diluted 1:5 in high-purity water) containing the Ligase enzyme. Following 6×10 min washes with Duolink In Situ Buffer A, the sections were incubated in a pre-heated humidity chamber for 100 min at 37° C. with Amplification Buffer (5× Duolink Amplification buffer diluted 1:5 in high-purity water) containing Polymerase enzyme (Duolink In Situ Detection Reagents Red, Sigma-Aldrich, DUO92008). The sections were then washed for 6×10 min with Duolink In Situ Buffer B at 20° C., followed by a 1-min wash in 0.01× Wash Buffer B. The sections were mounted with Sigma-Aldrich Duolink In Situ Mounting Medium containing 4′,6-diamidino-2-phenylindole (DAPI) (DUO82040). A single confocal plane consisting of individual tiles was captured using the Tiles tool of a Zeiss LSM800 AxioObserver Z1/7 confocal microscope with a Plan-Apochromatic 20×/0.8 M27 lens. For each fluorophore, all parameters (pinhole, excitation wavelength, emission power, and detector gain) were held constant for all imaging sessions. To correct for tiling artifacts that result from uneven illumination, an estimated shading profile was calculated for each channel with the Basic tool for illumination correction, using an ImageJ/Fiji Plugin (76) (https://github.com/marrlab/BaSiC). Shading correction was applied in Zen 2.6 software using the ‘Shading Correction’ function. In a subset of experiments when conjugating MINUS probe was unavailable from the manufacturer, the anti-KIBRA-MINUS probe was substituted with a biotinylated (Abcam Lightning-Link ab201795) anti-KIBRA ab216508 primary (1:400 in Duolink Antibody Diluent; incubated overnight at 4° C.). After 6×10 min washing (Duolink In Situ Buffer A), a goat anti-biotin secondary (1:200 in Antibody Diluent; Sigma B3640-1 MG) was applied for 2 h at 20° C. After another 6×10 min washing, the sections were incubated with Duolink PLA donkey anti-goat (DAG)-minus (1:5; DUO92003) in Antibody Diluent at 37° C. for 1 hr. Both methods yielded similar results.

### Immunocytochemistry

Hippocampal slices were fixed by immersion in ice-cold 4% paraformaldehyde in 0.1 M PB [pH 7.4] immediately after recording, and post-fixed for 48 h. Slices were then washed with PBS [pH 7.4] and cut into 20 μM sections using a Leica VT 1200S vibratome. Free-floating sections were permeabilized in 96-well plates with PBS containing 0.3% Triton X-100 (PBS-TX100) for 6×10 min at 20° C. and blocked with 10% normal donkey serum in PBS-TX100 for 2.5 h at 20° C. The sections were then incubated overnight at 4° C. with primary rabbit anti-KIBRA antibody (ab216508 Abcam, Waltham, MA) at 1:100 in PBS-TX100. After washing 6×10 min in PBS-TX100 at 20° C., the sections were incubated with Alexa Fluor 488-conjugated donkey anti-rabbit (1:200 in PBS-TX100; Jackson ImmunoResearch, West Grove, PA) for 2 h at 20° C. After 6×10 min washes in PBS-TX100 at 20° C., the slices were blocked for 2 h at 20° C. with 5% normal rabbit serum in PBS-TX100, followed by 6×10 min washes in PBS-TX100 at 20° C. The slices were then further blocked with 10% AffiniPure Fab Fragment donkey anti-rabbit IgG H+L (Jackson ImmunoResearch) PBS-TX100 for 2 h at 20° C. This additional blocking step was followed by 6×10 min washes in PBS-TX100 at 20° C. The sections were then incubated overnight at 4° C. with primary antibody rabbit anti-PKCζ/PKMζ C2 (1:4,000 (19); generated as previously described (12)) in PBS-TX100. After 6×10 min washes in PBS-TX100 at 20° C., the sections were incubated with Alexa Fluor 647-conjugated donkey anti-rabbit (1:200 in PBS-TX100; Jackson ImmunoResearch) for 2 h at 20° C. After washing 6×10 min in PBS-TX100, the sections were incubated with streptavidin conjugated-Alexa 647 (1:250 in PBS-TX100; Jackson ImmunoResearch) for 2 h at 20° C. After 6×10 min washes at 20° C. with PBS-TX100 and 10 min with PBS, the sections were mounted with DAPI Fluoromount-G (Southern Biotech). This procedure produces no bleedthrough between KIBRA and PKMζ fluorescent signals (FIG. 2B). A single confocal plane consisting of individual tiles was captured using the Tiles tool of a Zeiss LSM800 AxioObserver Z1/7 confocal microscope with a Plan-Apochromatic 20×/0.8 M27 lens. For each fluorophore, all parameters (pinhole, excitation wavelength, emission power, and detector gain) were held constant for all imaging sessions. To correct for tiling artifacts that result from uneven illumination, an estimated shading profile was calculated for each channel with the BaSiC tool for illumination correction, using an ImageJ/Fiji Plugin (76) (https://github.com/marrlab/BaSiC). Shading correction was applied in Zen 2.6 software using the ‘Shading Correction’ function.

### 3D Protein Modeling

Molecular graphics performed with UCSF ChimeraX (ver. 1.6.1), developed by the Resource for Biocomputing, Visualization, and Informatics at the University of California, San Francisco, with support from National Institutes of Health R01.GM129325 and the Office of Cyber Infrastructure and Computational Biology, National Institute of Allergy and Infectious Diseases (77, 78). The simulated protein models of KIBRA and PKMζ for illustration are developed by ModBase (79) and Tongil Ko at the University of Pennsylvania, respectively.

### BiFC Assay

### Transfection Constructs

KIBRA-PKMζ BiFC was performed as previously described (28), using pVen1-FLAG-KIBRA and pVen2-HA-PKMζ, in which PKMζ was cloned between EcoR1 and BamH1 sites. The constructs pVen1 and pVen2 encode the N-terminus (amino acids 1-154) and C-terminus (amino acids 155-238) of the Venus protein, respectively, and the Venus fragments were on the N-terminal of the fusion proteins. pVen2-HA-PKMζ[PKCι/λ-P291Q;F297S] was generated by site-directed mutagenesis using forward-cctggagAagcAAatceggatcccccggtCcctgtccgte and reverse-gacggacaggGaccgggggatccggatTTgctTctccagg primers and the QuickChange II XL Site-Directed Mutagenesis Kit (Agilent Technologies), following manufacturer's instructions. Additional pVen2-HA-PKC isoforms and pVen2-HA-CaMKIIα in the pVen2-HA vector were generated by cloning the N-terminus of the coding sequence of each kinase (table S1) to obtain an in-frame fusion at the C-terminus of the pVen2-HA. Human versions of KIBRA and kinases were used, and all PCR amplified sequences and constructs were verified by DNA sequencing.

### Cell Culture and Transfection

HEK293T cells were cultured in Dulbecco's Modified Eagle's Medium supplemented with 10% heat-inactivated fetal bovine serum, 100 U/ml penicillin, 100 mg/ml streptomycin, 2 mM glutamine, and 10 mM HEPES in a T75 tissue culture flask with canted neck and ventilated cap. Twenty-four hours prior to transfection, 1×10⁵ HEK293T cells in 1 ml of media were plated on poly-D-lysine-treated coverslips in 24-well plates. One hour prior to transfection, 0.5 ml of the media was removed from each well, and ζ-stat or K-ZAP was added to the media that had been removed. The remaining media in each well was then discarded and substituted with the 0.5 ml pre-conditioned media containing ζ-stat or K-ZAP at designated concentrations. For transfection a total of 50 ng of DNA was delivered to each well using Lipofectamine 3000 (Invitrogen) and OptiMEM 1× reduced serum medium (Invitrogen) at a plasmid-to-plasmid ratio of 1:1. The amount of transfected DNA was optimized to produce relatively low concentrations of proteins during overexpression. The cells were co-transfected with pVen1-FLAG-KIBRA and pVen2-HA-PKM, as previously described (28), or pVen1-FLAG-KIBRA and pVen2-HA-PKMζ [PKC/A-P291Q;F297S]/PKC isoforms/CaMKIIα. As controls, cells were co-transfected with pVen1-FLAG-KIBRA and pVen2-HA, or pVen1-FLAG and pVen2-HA-PKMζ. After transfection, the cells were incubated for 24 h, fixed with 4% paraformaldehyde, and kept on 0.02% sodium azide in 1×PBS at 4° C. until immunostaining.

### Immunostaining and Confocal Microscopy

To detect FLAG and HA tags in cells transfected with BiFC plasmids, the cells were blocked and permeabilized using blocking buffer (2% bovine serum albumin [BSA], 0.05% Tween-20, 0.1% Triton X-100 in 1×PBS) for 15 min at 20° C. The primary antibodies, mouse anti-FLAG (1:100, Sigma Aldrich) and rabbit anti-HA (1:100, Sigma Aldrich), were diluted in blocking buffer. The blocking solution was then removed and 200 μl of the diluted primary antibody was added to each well, and the samples were incubated overnight at 4° C. The primary antibody was then removed, followed by 3×5 min washes with 0.05% Tween-20 in 1×PBS. The cells were then incubated for 1 h in the dark at 20° C. with secondary antibodies, Alexa Fluor 647-conjugated goat anti-mouse (1:250, Invitrogen) and Alexa Fluor 594-conjugated goat anti-rabbit (1:250, Invitrogen), diluted in blocking buffer. The secondary antibody solution was removed, and the same washing steps as with the primary antibody solution were performed. The cells were mounted on glass slides with DAPI Fluoromount-G (Southern Biotech). Z-stack images of random microscopic fields were acquired with a 1 μm z-step on a confocal microscope Zeiss LSM800 AxioObserver Z1/7 using a Plan-Apochromatic 63×/1.4NA oil objective and exported as maximum intensity projection. All imaging parameters (pinhole, excitation wavelength, emission power, and detector gain) were constant for all experimental conditions. To determine the number of interacting BiFC puncta per cell, the images were converted to greyscale and the ImageJ 1.52n (80) freehand or wand selection tools were used to create an outline of each cell, which was added to the region of interest (ROI) manager. To ensure that HEK293T cells were transfected with both pVen1-FLAG-KIBRA and pVen2-HA-PKMζ (or pVen1-FLAG-KIBRA and pVen2-HA-PKMζ[PKCι/λ-P291Q;F297S]/PKC isoforms/CaMKIIα), only cells that were positive for FLAG and HA tags signals were included in the analysis. The “find maxima” algorithm from ImageJ was used to count the number of local maxima per cell with a noise tolerance of 10, as previously described (87). Individual data points for each experimental condition (control and treatment) consist of the means of >50 cells obtained from two independent cultures.

### Behavior—Active Place Avoidance

### Intrahippocampal Injection

For spatial long-term memory experiments, we adapted the approach used in Garcia-Osta et al. (82). Mice were ˜12-weeks old at surgery. Briefly, to implant the injection cannula hardware, mice were anesthetized by an intraperitoneal (i.p.) injection of a mixture of dexmedetomidine (5 mg/kg body weight) and ketamine (28 mg/kg body weight), and mounted in a Kopf stereotaxic frame (Tujunga, CA). The tips of guide cannula (Plastics One, Roanoke, VA; Part Numbers: C235GS-5-2.0) were targeted above the injection target in the dorsal hippocampus (AP-1.94 mm; ML±1.00 mm; DV-0.90 mm). The other injection hardware (Part Numbers: C235DCs-5, 303DC/1; cannula dummy, cannula cap, respectively) was assembled, and antisedan (0.65 mg/kg body weight i.p.) was administered to reverse the sedation at the end of surgery.

Three to four weeks after surgery, the animals received active place avoidance training. Before testing the effect of the drug injection on place avoidance, the animals received a bilateral injection of vehicle (PBS [pH 7.4] 0.5 μl/side) and were left in the home cage to habituate to the procedure. Depending on the experimental design, injection of vehicle or drug, either ζ-stat or K-ZAP (5 nmol in 0.5 μl PBS/side), is 1 day or 4 weeks after the training session. During the injection, the animals were restrained, the cannula cap and dummy removed, and the injection needle (Plastics One, Roanoke, VA; Part Numbers: C235IS-5) inserted into the guide cannula so that it protruded from the end of the guide by 0.5 mm. The other end of the needle was connected to a 10 μl Hamilton syringe via Tygon tubing. The drug or vehicle was infused for 1 min, and after the infusion the needle was left in place for 5 min before removal. The animals were then returned to their home cages until the memory retention tests that were conducted 2 days later.

### Conditioning

Active place avoidance was conducted with a commercial computer-controlled system (Bio-Signal Group, Acton, MA). The mouse was placed on a 40-cm diameter circular arena rotating at 1 rpm. The specialized software, Tracker (Bio-Signal Group, Acton, MA), was used to detect the animal's position 30 times per second by video tracking from an overhead camera. A clear wall made from polyethylene terephthalate glycol-modified (PET-G) was placed on the arena to prevent the animal from jumping off the elevated arena surface. A 5-pole shock grid was placed on the rotating arena, and the shock was scrambled across the 5-poles when the mouse entered the shock zone. All experiments used the “Room+Arena−” task variant that challenges the mouse on the rotating arena to avoid a shock zone that was a stationary 60° sector (7). Every 33 ms, the software determined the mouse's position, whether it was in the shock zone, and whether to deliver shock. After the animal enters the shock zone for 500 ms, a constant current foot-shock (60 Hz, 500 ms) was delivered and repeated with the interval of 1500 ms until the mouse left the shock zone. The shock intensity was 0.2 or 0.3 mA, which was the minimum amplitude to elicit flinch or escape responses. The animal was forced to actively avoid the designated shock zone because the arena rotation periodically transported it into the shock area.

The tracked animal positions with timestamps were analyzed offline (TrackAnalysis, Bio-Signal Group, Acton, MA) to extract several end-point measures. The time to first enter the shock zone estimates ability to avoid shock and was taken as an index of between-session memory. The number of entrances within one trial was taken as another index to examine the animal learning curve throughout all training trials. A pretraining habituation period on the apparatus equivalent in time to a training session, but without shock, was provided.

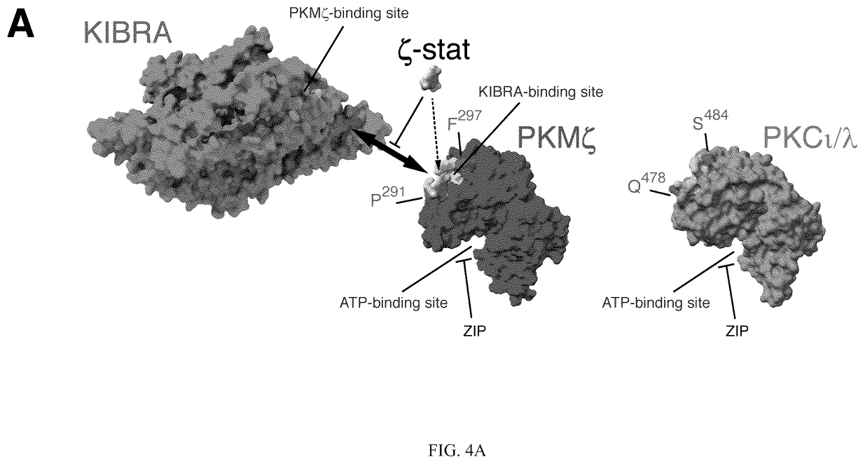

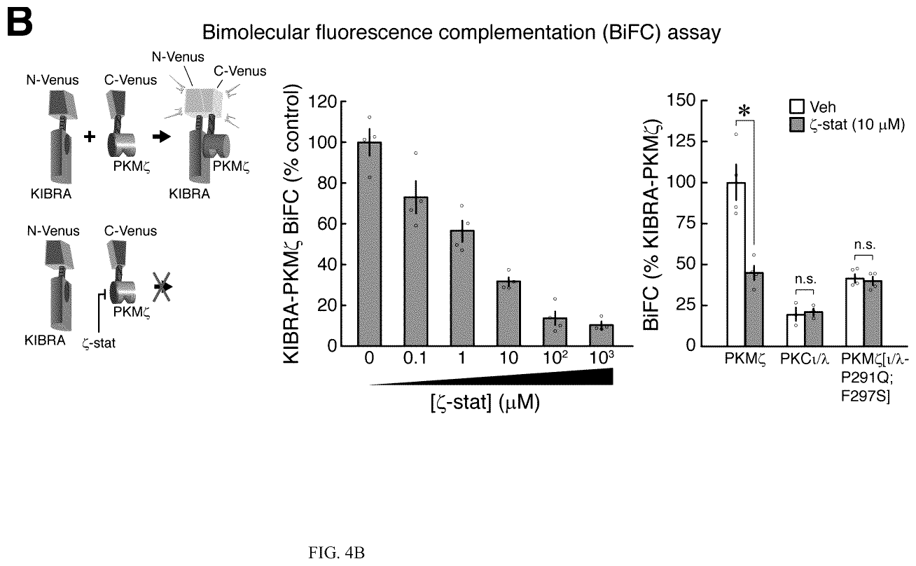

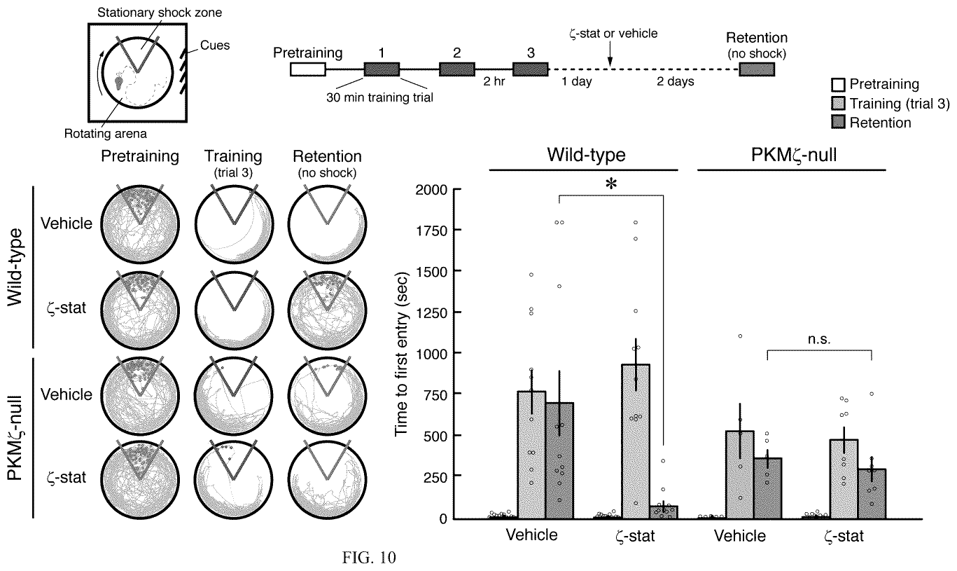

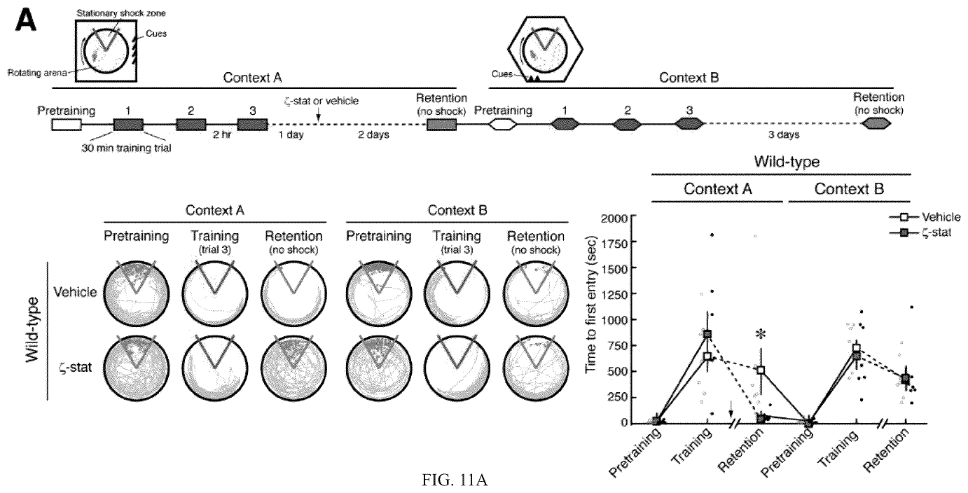

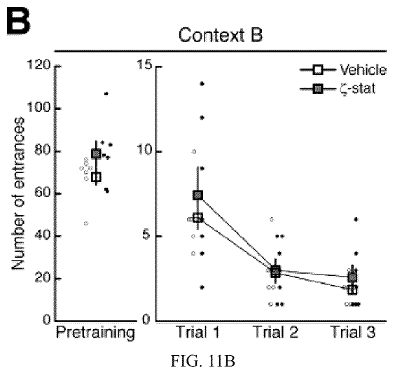

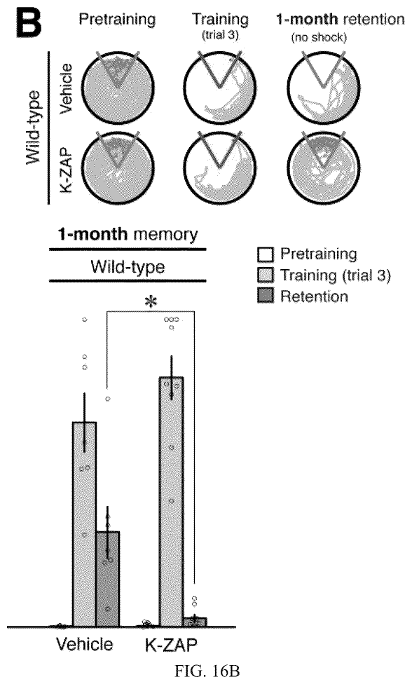

The training schedule was as follows: 2 h after a 30-min pretraining habituation, the animals received three 30-min training trials, with an intertrial interval of 2 h. Bilateral intrahippocampal injection of ζ-stat or vehicle (FIG. 10), or K-ZAP or vehicle (FIG. 16) was 24 h (FIG. 10 and FIG. 16A) or 4 weeks (FIG. 16B) after the training session. Retention testing was a 30 min trial without shock 2 days after injection. In FIG. 11, the animals first received the conditioning in Context A, injection, and a memory retention test as described above. Immediately after retention testing, the animals were conditioned in Context B with a different set of spatial cues, and memory for active place avoidance in Context B tested 3 days later. Pre-established exclusion criterion was if after sacrificing the animals and performing histology for cannula placement, cannulae were found to be incorrectly targeted. The data from 2 animals were excluded from behavioral analysis of the effects of ζ-stat on PKMζ-null mice because histology revealed misplaced cannulae.

Behavior-Auditory-cued fear/threat conditioning

### Animals

Mice were 8-10 weeks old at the time of cannulation and 9-11 weeks old at the beginning of behavioral experiments. Mice were housed with cage-mates in plastic cages and provided with food and water ad libitum. Mice were maintained on a 12 h light/dark cycle (lights on at 7:00 am) and behavioral experiments began at 9:00 am.

### Surgery

Mice were injected intraperitoneally (1 ml/100 g body weight) with an anesthetic cocktail containing ketamine (10 mg/ml) and xylazine (2 mg/ml). Mice were provided with analgesic treatment prior to surgery (carprofen; 5 mg/ml). Guide cannulae (Plastics One, Roanoke, VA) were implanted bilaterally in the basolateral amygdala (from bregma: AP-1.7 mm; L+/−3.0 mm; DV-4.4 mm) and secured to the skull with three jeweler's screws and dental cement. Antisedan (0.66 ml/100 g body weight of 0.5 mg drug/ml solution) was given via i.p. injection after surgery to reverse the anesthesia.

### Conditioning

For 7 days following surgery, mice were handled by freely exploring the experimenter's palm for 2-5 min. Mice were habituated, trained, and tested in the same conditioning box (Coulbourn Habitest, Coulboum Instruments) with differing floors and walls to produce two different contexts (Context A and Context B). For each day of the behavioral experiment, mice were brought to the experiment room at 9:00 am and allowed to acclimatize for 30 min. Mice were then habituated to the testing context (Context A with smooth floor and flat, blank walls) for 20 min each day for two consecutive days. The next day, mice were trained in a second context (Context B, with a grid floor, patterned walls, and a curved wall). Training consisted of 2 min of exploration of Context B followed by a tone (2800 Hz, 85 dB, 30 s) co-terminating with a footshock (0.7 mA, 1 s). Mice received two tone-shock pairings separated by 1 min and remained in Context B for an additional 1 min before returning to their home cage. Mice were tested 24 h after training in Context A (Test 1). During testing, mice were placed in the conditioning box and, after 2 min, were exposed to a 30 sec tone (2800 Hz, 85 dB). The next day, mice received bilateral infusions and were tested a second time 24 h post-infusion in Context A (Test 2). Freezing behavior (cessation of all movement except breathing) during the tone on Test 1 and Test 2 was scored by an experimenter blind to the conditions. Scores are reported as the percent of time spent freezing during the tone. Pre-established exclusion criteria were: 1) if the mouse froze less than 25% of the time after the conditioned stimulus was presented at the first test, which is the standard cut off used in the Nader lab to distinguish mice that had learned or not learned the conditioned stimulus-unconditioned stimulus association; 2) if after sacrificing the animals and performing histology for cannula placement, cannulae were found to be incorrectly targeted.

### Drug Infusions

Mice were bilaterally infused with ζ-stat obtained from Drug Synthesis and Chemistry Branch, National Cancer Institute, or vehicle (PBS [pH 7.4]). Mice were infused with 6 nmol ζ-stat in 0.3 μl at a rate of 0.2 μl/min into each basolateral amygdala. Drugs were infused with 28-gauge microinjectors (Plastics One, Roanoke, VA) connected to Hamilton syringes (26 gauge, Model 170 IN) by way of polyethylene tubing (Braintree Scientific, Inc., Braintree, MA). After infusion, injectors remained in place for 1 min to ensure drug diffused sufficiently away from the injector tip.

### Behavior-Contextual Fear/Threat Conditioning

### Animals

Mice were 8-10 weeks old at the time of cannulation and 9-11 weeks at the beginning of behavioral experiments. Mice were housed with cage-mates in plastic cages and provided with food and water ad libitum. Mice were maintained on a 12 h light/dark cycle (lights on at 7:00 am) and behavioral experiments began at 9:00 am.

### Surgery

Mice were injected intraperitoneally (1 ml/100 g body weight) with an anesthetic cocktail containing ketamine (10 mg/ml) and xylazine (2 mg/ml). Mice were provided with analgesic treatment prior to surgery (carprofen; 5 mg/ml). Guide cannulae (Plastics One, Roanoke, VA) were implanted bilaterally in the dorsal hippocampus (from bregma: AP-2.1 mm; L+/−1.8 mm; DV-1.2 mm) and secured to the skull with three jeweler's screws and dental cement. Antisedan (0.66 ml/100 g body weight of 0.5 mg drug/ml solution) was given via i.p. injection after surgery to reverse the anesthesia.

### Conditioning

For 7 days following surgery, mice were handled by freely exploring the experimenter's palm for 2-5 min. Mice were trained and tested in the same conditioning box (Coulbourn Habitest, Coulboum Instruments), which consisted of a grid floor and blank walls. For each day of the behavioral experiment, mice were brought to the experiment room at 9:00 am and allowed to acclimatize for 30 min. On the first day (training), mice explored the context for 2 min. We then delivered two 0.7 mA (1 s) shocks spaced 1 min apart. Mice remained in the box for an additional minute before returning to their home cage. The next day, mice received bilateral infusions and were tested 24 h post-infusion. During testing, mice were placed in the same context for 4 min with no shock. Freezing behavior (cessation of all movement except breathing) during the 4 min session was recorded by an experimenter blind to the conditions. Scores are reported as the percent of time spent freezing during the 4 min session. Pre-established exclusion criterion was if after sacrificing the animals and performing histology for cannula placement, cannulae were found to be incorrectly targeted.

### Drug Infusions

Mice were bilaterally infused with vehicle (PBS [pH 7.4]) or ζ-stat (10 nmol in 0.5 μl per hemisphere at a rate of 0.2 μl/min/side). Drugs were infused with 28 gauge microinjectors (Plastics One, Roanoke, VA) that protruded 0.3 mm from the guide cannulae, connected to Hamilton syringes (26 gauge, Model 1701N) by way of polyethylene tubing (Braintree Scientific, Inc., Braintree, MA). After infusion, injectors remained in place for 1 min to ensure drug diffusion away from the injector tip.

### Immunoblotting

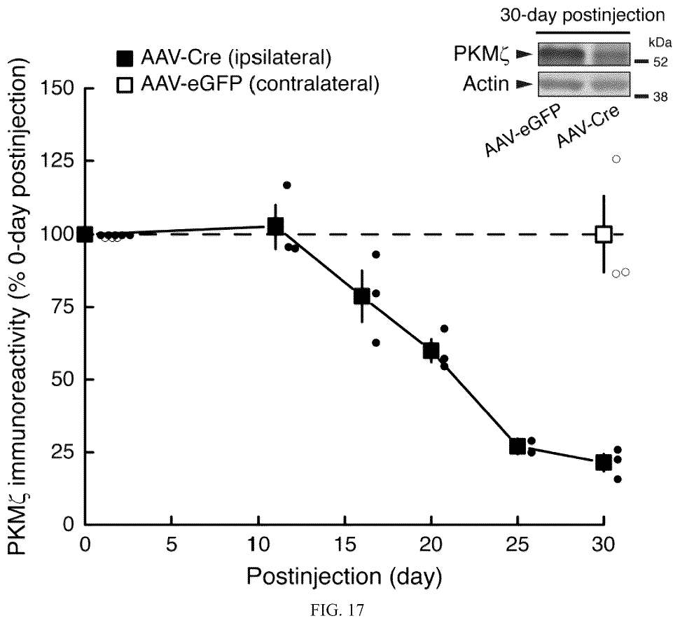

Immunoblots for FIG. 17 were performed as previously described (19).

### Statistical Analysis

Replicates are biological because there is only one measurement for each sample. Drug/vehicle comparisons were performed blindly. Sample sizes vary for the different experimental approaches (biochemistry, slice physiology, and behavior) and were established by power analyses based on effect size estimates from published work or preliminary experiments. The observed effect sizes for binary comparisons are reported as Cohen's d and as η²ₚ for ANOVA effects. Multi-factor comparisons were performed using ANOVA with repeated measures, as appropriate. Two-population Student's/tests were performed to compare protein immunofluorescence intensity between control and potentiated hippocampal slices. For LTP experiments, the responses were first normalized to the mean of the 30 min period prior to tetanization (or equivalent in control pathways), and then the means over 5 min periods were used for statistical comparisons (e.g., pre-tetanization, beginning and end of drug application). Paired Student's t tests were used to compare the change in the potentiated response at time points at the beginning and end of drug application. The degrees of freedom for the critical t values of the/tests and the F values of the ANOVAs are reported as subscripts. Post-hoc multiple comparisons were performed by Newman-Keuls tests as appropriate. Statistical significance was accepted at P<0.05 or appropriate Bonferroni correction for multiple comparisons.

### Example 1: KIBRA-PKMζ Complexes Persistently Increase in LTP Maintenance

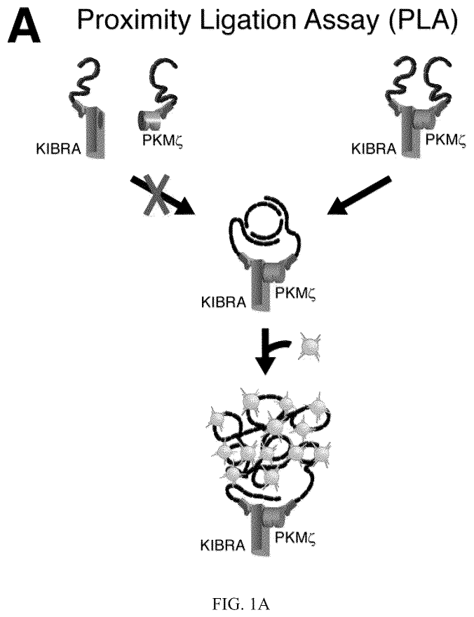

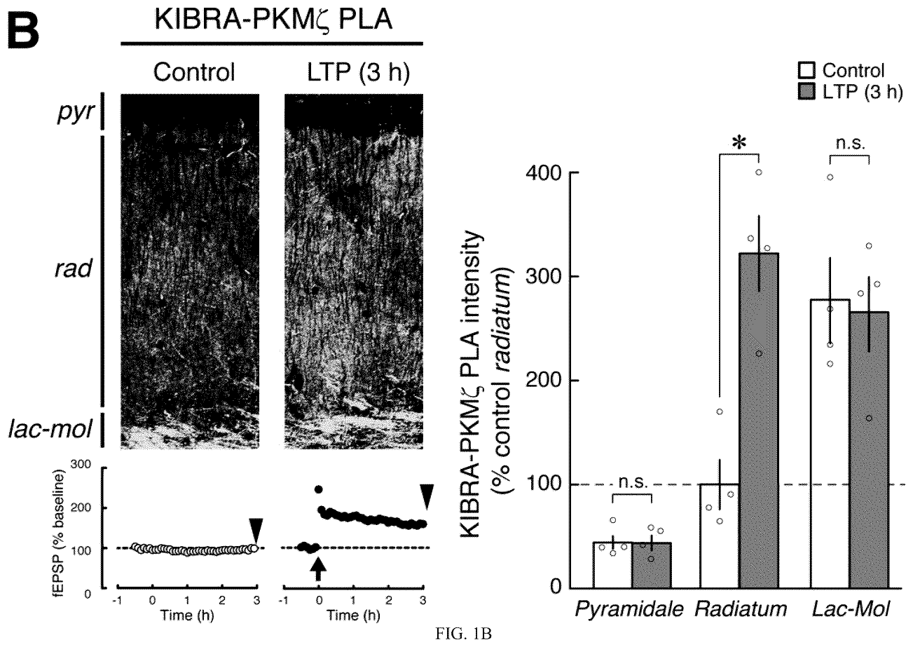

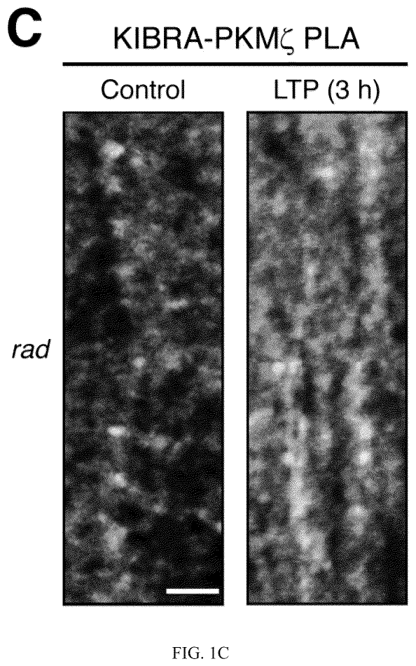

We used in situ proximity ligation assay (PLA) to detect molecular complexes of KIBRA and PKMζ in late-LTP maintenance (FIG. 1 and FIG. 2A). In PLA, pairs of antibodies are linked to oligonucleotides, and if the molecules recognized by the antibodies are within 40 nm, the oligonucleotides generate a DNA that is amplified and detected by fluorescent probes (FIG. 1A) (41). To establish late-LTP, we prepared mouse hippocampal slices and briefly tetanized Schaffer collateral/commissural fibers in CA3 stratum (st.) radiatum to potentiate field excitatory postsynaptic potential (fEPSP) responses recorded in CA1 st. radiatum for 3 h (42) (FIG. 1B). As control, we recorded low-frequency test responses in an adjacent slice from the same hippocampus for the equivalent time. The strong stimulation facilitates persistent increases of KIBRA-PKMζ complexes in st. radiatum (FIG. 1B), which accumulate in ˜1 μm puncta along CA1 pyramidal cell dendrites (FIG. 1C). In contrast, the complexes do not increase in st. lacunosum-moleculare that receive unstimulated synaptic projections, despite high levels of complexes under basal conditions in this dendritic region. The relatively few complexes in CA1 pyramidal cell bodies of st. pyramidale also do not increase.

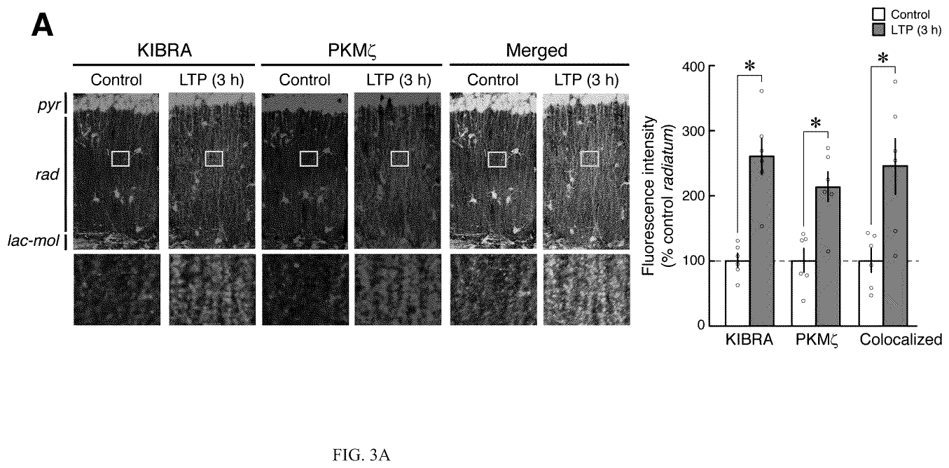

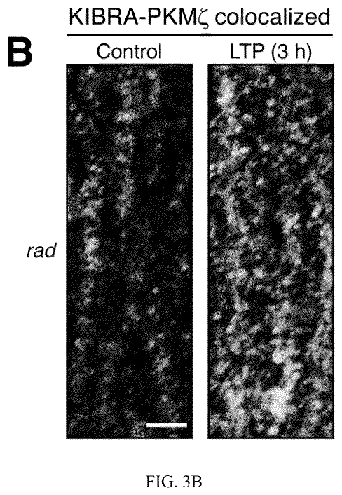

In parallel with PLA, we examined KIBRA and PKMζ colocalization a second way by measuring the proteins individually by immunocytochemistry (FIG. 3 and FIG. 2B). Stimulation persistently increases the amounts and colocalization of KIBRA and PKMζ in st. radiatum (FIG. 3A). The colocalized proteins accumulate in ˜1 μm puncta along CA1 pyramidal cell dendrites (FIG. 3B). Comparing immunocytochemistry and PLA in CA1 pyramidal cell bodies, however, reveals high levels of KIBRA and PKMζ yet low levels of KIBRA-PKMζ complexes; therefore, somatic KIBRA and PKMζ do not appear to interact (FIG. 1B and FIG. 3A). Thus, strong afferent synaptic activity acts locally within neurons to persistently increase KIBRA-PKMζ interaction.

### Example 2: Blocking the KIBRA-Binding Site in PKMζ Reverses Late-LTP Maintenance

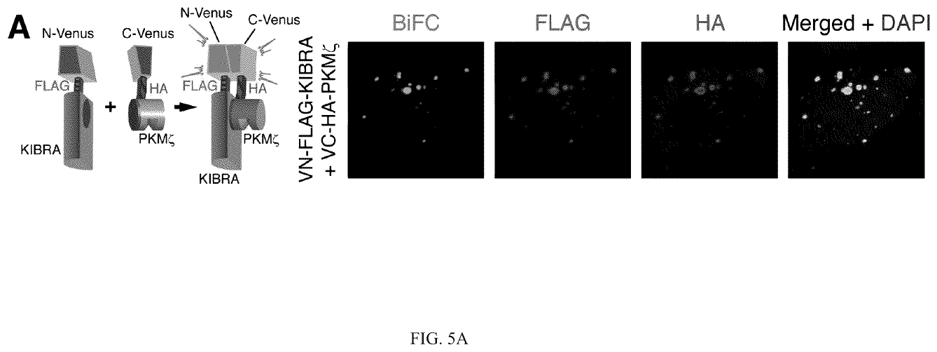

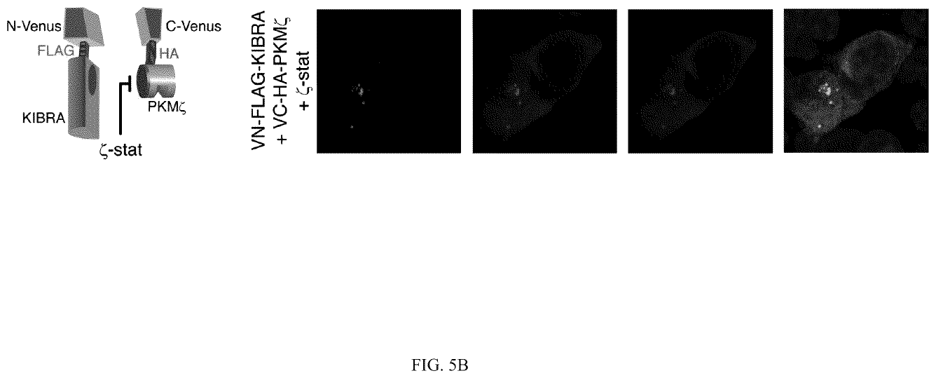

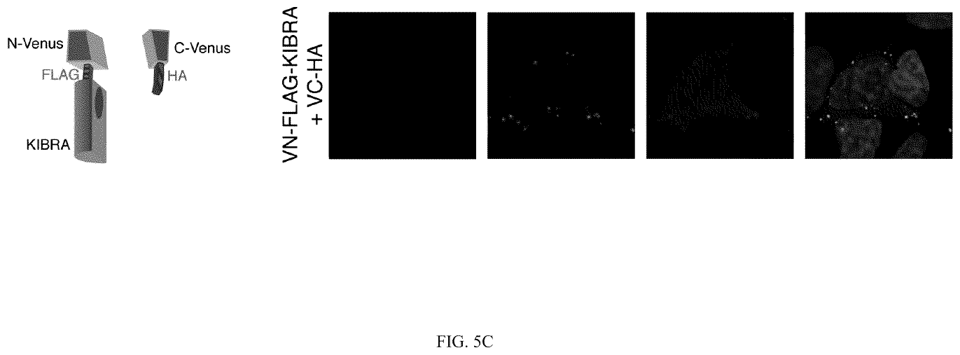

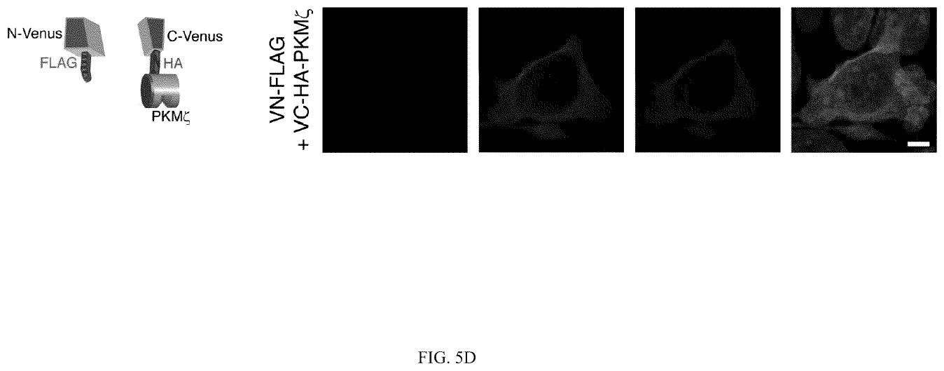

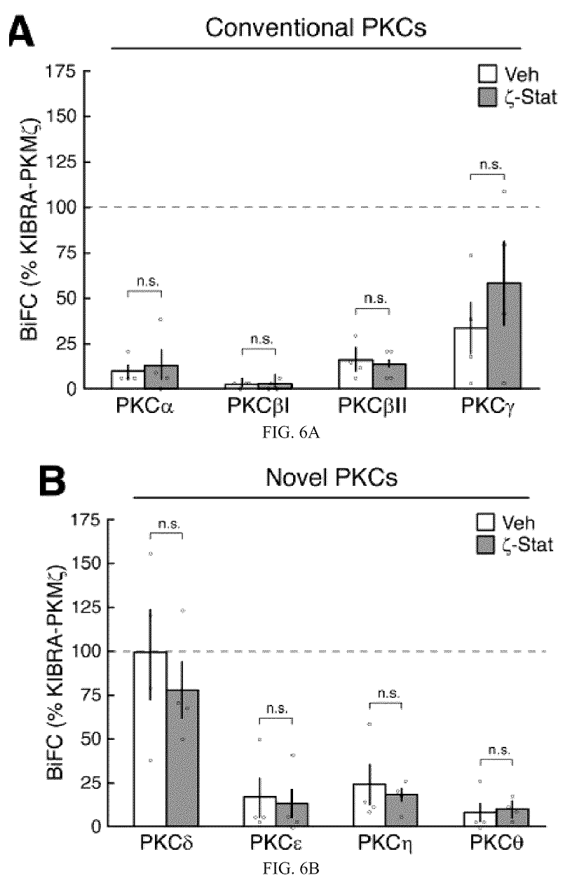

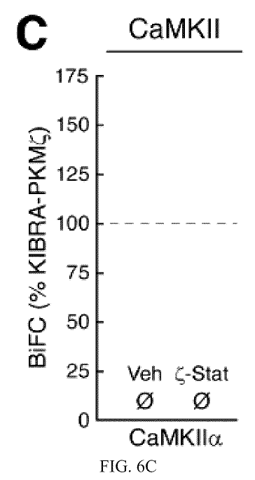

As most signaling events triggered by strong afferent synaptic activity last for only seconds to minutes (11, 43-45), the persistence of KIBRA-PKMζ complexes for hours in late-LTP suggests that sustained KIBRA-PKMζ interaction might maintain late-LTP in wild-type mice. To test this “KIBRA-PKMζ maintenance” hypothesis, we used the small molecule PKMζ-inhibitor, C-stat (1-naphthol-3,6,8-trisulphonic acid), which has been proposed to selectively block the allosteric KIBRA-binding site in the ζ-catalytic domain (37) (FIG. 4A). To assess this mechanism of action we isolated KIBRA-PKMζ heterodimerization/multimerization using the split-Venus bimolecular fluorescence complementation reporter assay (BiFC) (28) (FIG. 4B). In this assay KIBRA and PKMζ fused with complementary fragments of the fluorescent reporter Venus are transfected into HEK293T cells, and the interaction of KIBRA and PKMζ produces a fluorescent signal by bringing the two fragments of split-Venus into close apposition (28) (FIG. 4B, left, FIG. 5). BiFC reveals G-stat inhibits KIBRA-PKMζ interaction (IC₅₀=˜1 μM, FIG. 4B, middle). In contrast, the C-stat at 10 μM has no measurable effect on the interaction of KIBRA with the other atypical isoform, PKCι/λ, which produces BiFC with KIBRA ˜10-fold less than PKMζ (FIG. 4B, right). We further validated ζ-stat's mechanism of action by generating a mutant PKMζ[PKCι/λ-P291Q;F297S] with the C-stat-binding site in PKMζ changed to the analogous amino acids in PKCι/λ. Interaction of KIBRA with PKMζ[PKCι/λ-P291Q;F297S] is similar to PKCι/λ, and, like PKCι/λ, ζ-stat does not inhibit complexes of mutated PKMζ with KIBRA. ζ-stat also has no effect on KIBRA's interaction with any conventional or novel PKC, the two other classes of PKC isoforms (FIGS. 6, A and B). Ca²⁺/calmodulin-dependent protein kinase II alpha (CaMKIIα), another kinase that potentiates synaptic transmission and is important for early-LTP (44, 46), does not measurably interact with KIBRA (FIG. 6C).

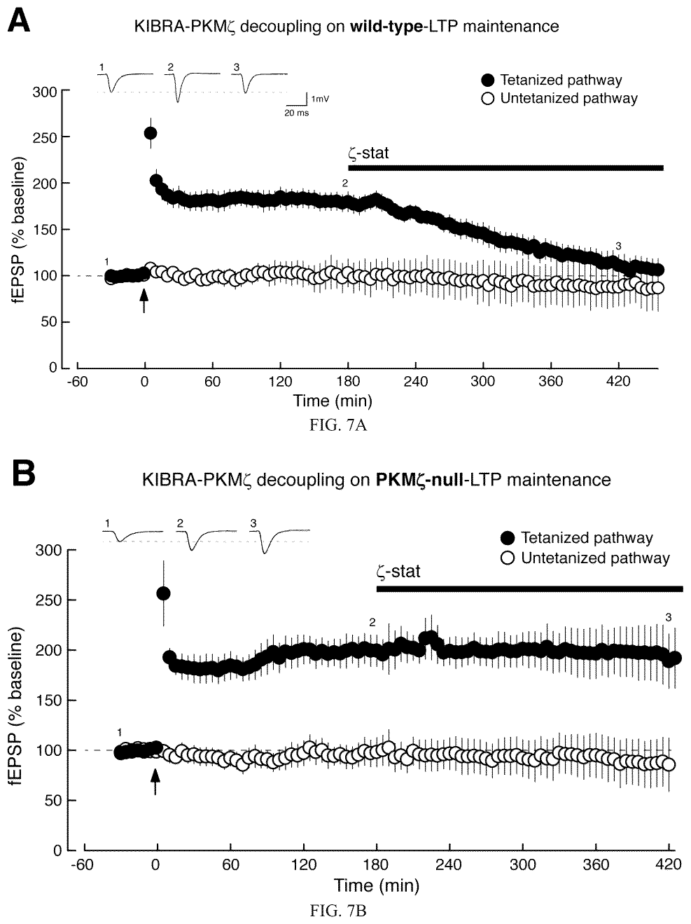

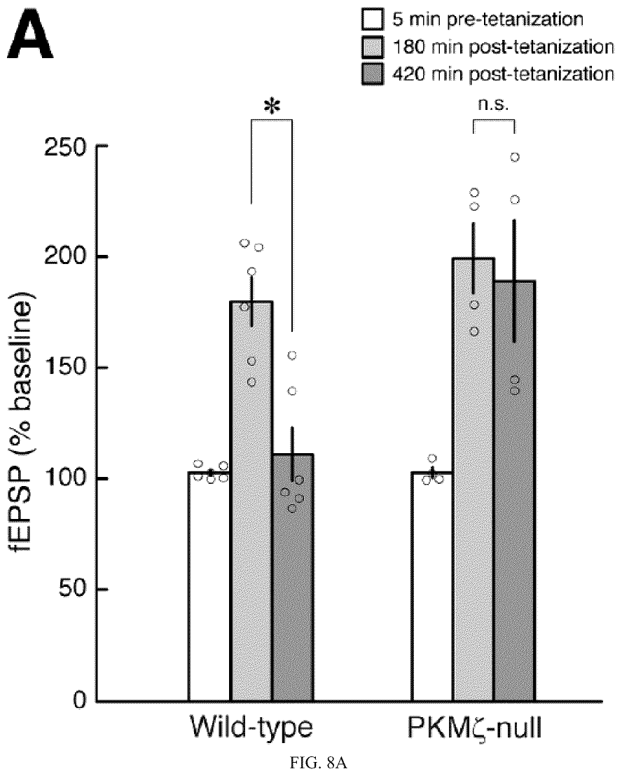

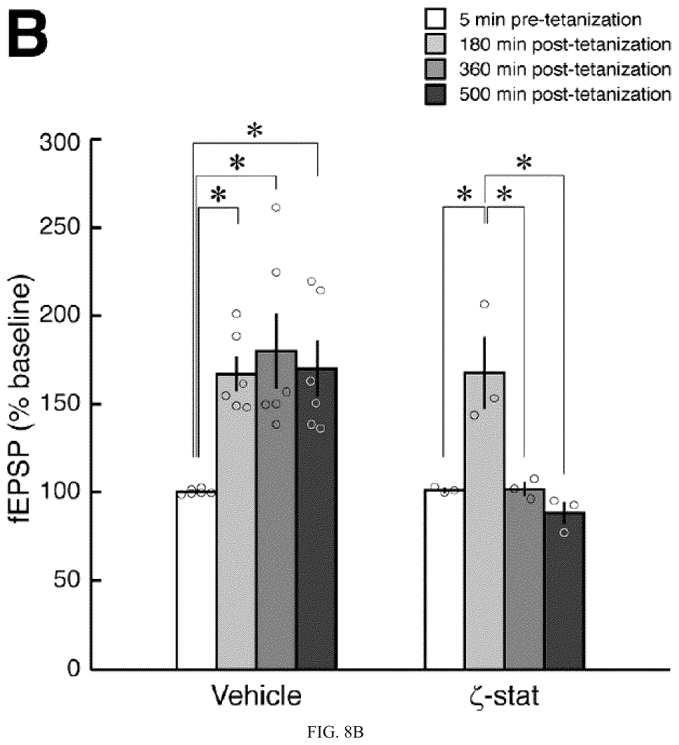

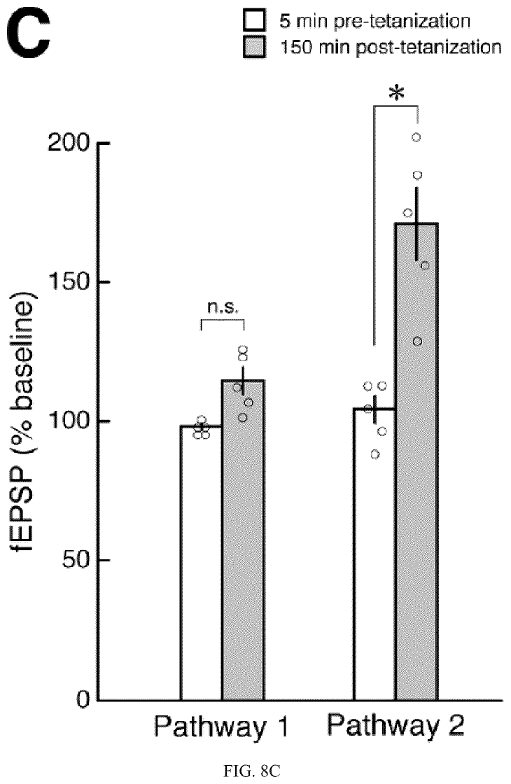

Now we can test the central prediction of the KIBRA-PKMζ maintenance hypothesis that decoupling PKMζ from KIBRA reverses potentiation of activated synapses. We simultaneously recorded two independent synaptic pathways in st. radiatum within a hippocampal slice. We stimulated one pathway by high-frequency tetanization to induce late-LTP, and in the second pathway we recorded low-frequency test fEPSP responses for the equivalent time. After establishing late-LTP for 3 h, we applied 10 μM ζ-stat to the bath and recorded responses in both pathways for 4 h (FIG. 7A and FIG. 8A). ζ-stat reverses late-LTP maintenance in the stimulated synaptic pathway without measurably affecting synaptic transmission in the control pathway. Thus, ζ-stat disrupts the maintenance of enhanced synaptic transmission selectively at activated synapses, as predicted by the KIBRA-PKMζ maintenance hypothesis.

We tested whether the effect of ζ-stat requires PKMζ by examining PKMζ-null mice that recruit compensatory PKMζ-independent mechanisms of late-LTP (17). In striking contrast to wild-type mice, ζ-stat has no effect on late-LTP maintenance in mice lacking PKMζ (FIG. 7B and FIG. 8A).

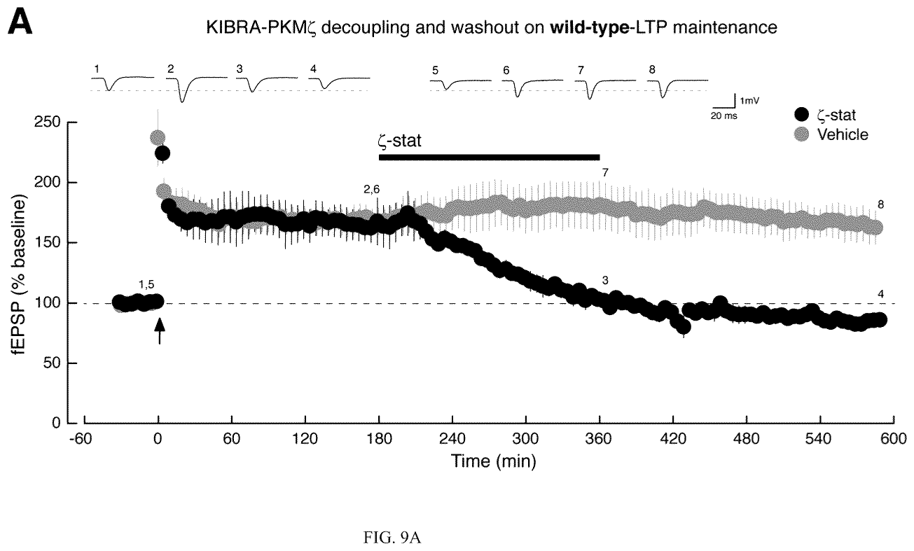

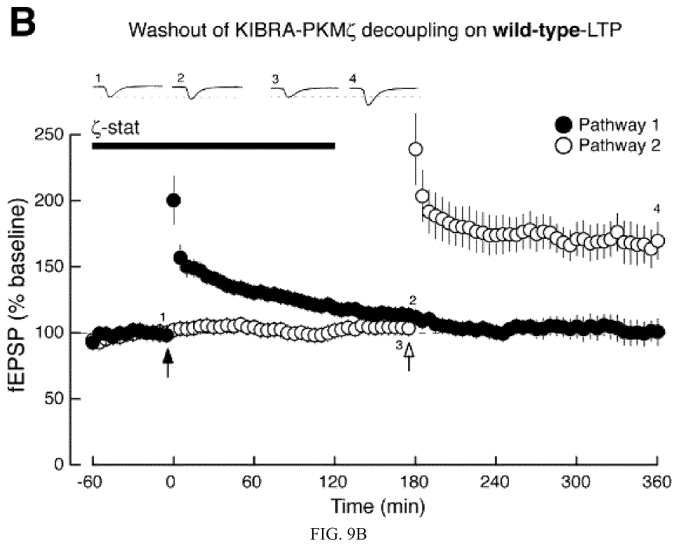

If KIBRA-PKMζ coupling in wild-type mice maintains LTP, then when ζ-stat is washed out the reversal of potentiation should persist. If another mechanism maintains LTP, and KIBRA-PKMζ coupling is a transient, downstream signaling pathway that expresses synaptic potentiation, then when the drug is washed out potentiation should return. To distinguish between the KIBRA-PKMζ maintenance hypothesis and this alternative KIBRA-PKMζ downstream-expression hypothesis, after establishing wild-type late-LTP, we applied ζ-stat for 3 h and then washed the drug out for an additional 4 h. The reversal of potentiation persisted (FIG. 9A and FIG. 8B). To examine whether the washout was effective, in separate experiments we applied the drug for the same 3 h duration, suppressing late-LTP in one synaptic pathway, and then initiated the washout (FIG. 9B and FIG. 8C). After only 1 h of washout, we stimulated a second synaptic pathway and produced late-LTP, indicating effective drug washout. These results are predicted by the KIBRA-PKMζ maintenance hypothesis and not by the KIBRA-PKMζ downstream-expression hypothesis.

### Example 3: Blocking the KIBRA-Binding Site in PKMζ Disrupts Established Long-Term Memory

We next tested the predictions of the KIBRA-PKMζ maintenance hypothesis in active place avoidance, a hippocampus-dependent spatial long-term memory task (FIG. 10). We rapidly conditioned wild-type mice to actively avoid a shock zone by three 30-min training sessions with 2-hour intertrain intervals, and then 1 day later injected ζ-stat or vehicle bilaterally in hippocampus. Two days after the injection, we tested long-term memory retention with the shock off. Mice that had received vehicle remember to avoid, whereas mice that had received ζ-stat show persistent loss of retention for the shock zone location. In contrast, PKMζ-null mice receiving ζ-stat avoided the shock zone as well as those that had received vehicle, thus controlling for off-target effects of the drug in memory maintenance.

We examined whether C-stat disrupts maintenance as opposed to other aspects of memory (FIG. 11A). Repeating the experiments in wild-type mice, we measured the persistent loss of long-term memory retention without shock and then immediately retrained the mice to avoid another shock zone defined by the cues of a novel context. The mice that had previously been injected with ζ-stat do not remember the location of the first shock zone that was established before the injection, but they learn to avoid and remember a new second location as well as the mice that had received only vehicle. Indeed, the mice that had earlier received ζ-stat have learning curves in the second context indistinguishable from vehicle-treated mice, demonstrating ζ-stat does not suppress expression of the avoidance behavior (FIG. 11B). Thus, C-stat persistently disrupts previously acquired information, but once eliminated, does not impair formation, maintenance, or expression of newly acquired spatial information.

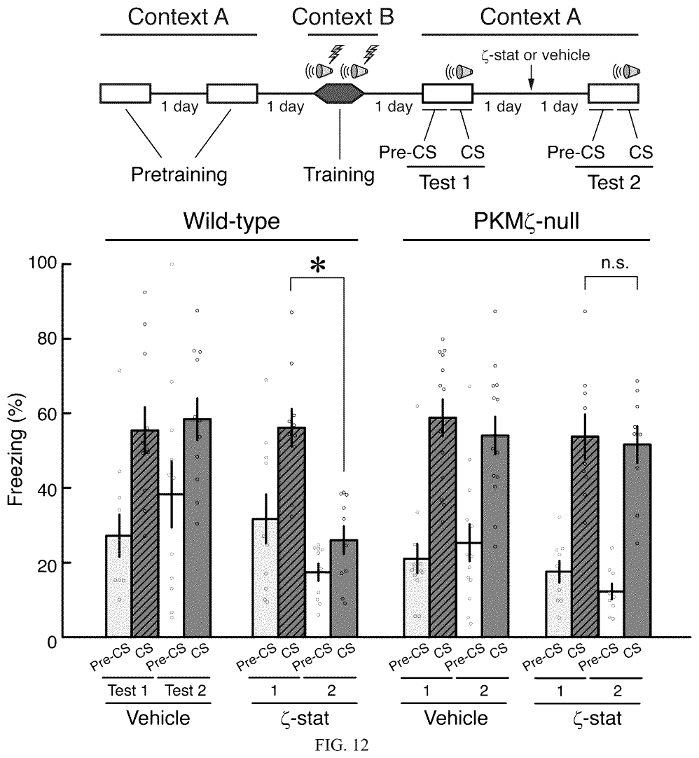

We also examined the consequences of KIBRA-PKMζ decoupling on auditory-cued fear/threat memory maintenance in the basolateral amygdala (BLA) of wild-type and PKMζ-null mice (FIG. 12). One day after conditioning, both wild-type and PKMζ-null mice show increased freezing to tone, confirming previous findings in PKMζ-null mice (23). Then 1 day after the first memory retention testing, the mice were injected with ζ-stat or vehicle bilaterally in BLA and retested the following day. The results reveal that ζ-stat impairs memory retention in wild-type mice and not in PKMζ-null mice. We note that the ζ-stat treatment in wild-type mice results in freezing that is indistinguishable from freezing before presentation of the conditioned stimulus (for ANOVA see FIG. 12 legend; post hoc analysis shows P=0.34).

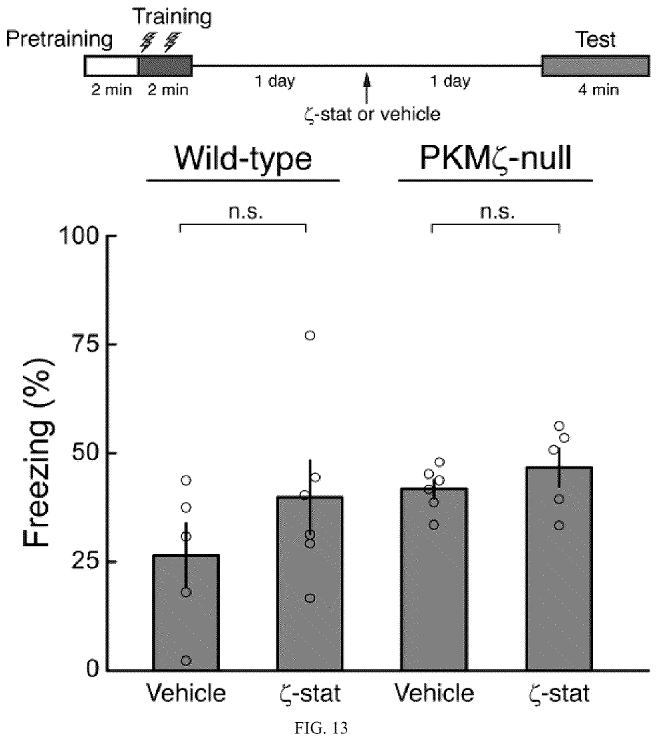

As PKMζ-null mice show a form of PKMζ-independent memory maintenance, we asked whether wild-type mice might as well. Previous results with the first-generation PKMζ-inhibitor ZIP revealed that whereas the agent disrupts cued fear/threat memory, ZIP in hippocampus does not affect contextual fear/threat memory (47, 48). We found that like ZIP, C-stat has no effect on contextual fear/threat memory in hippocampus, either in wild-type mice or PKMζ-null mice (FIG. 13). Therefore, wild-type mice store information by both PKMζ-dependent mechanisms and, like PKMζ-null mice, PKMζ-independent mechanisms.

### Example 4: Peptide Mimicking the PKMζ-Anchoring Site in KIBRA Reverses Late-LTP Maintenance and Disrupts Long-Term Memory

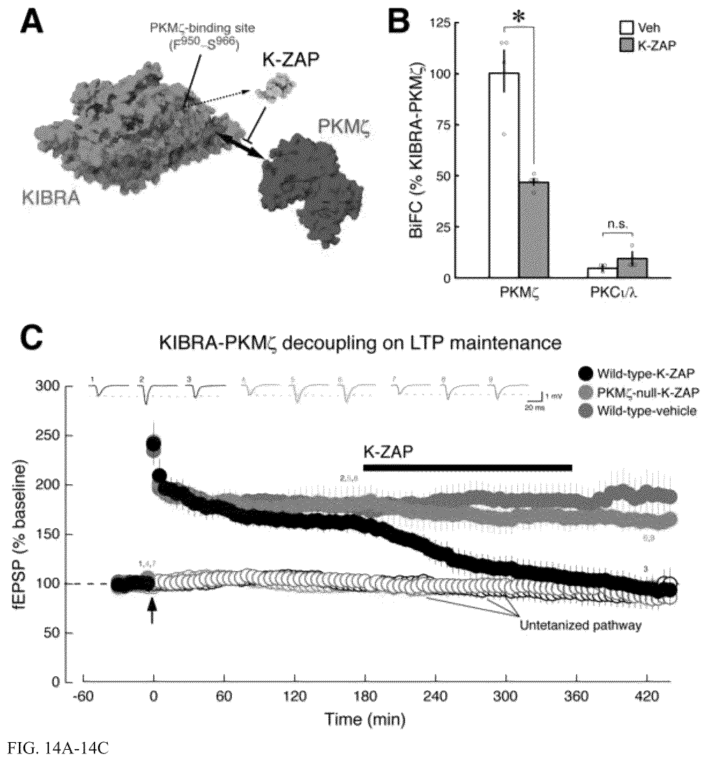

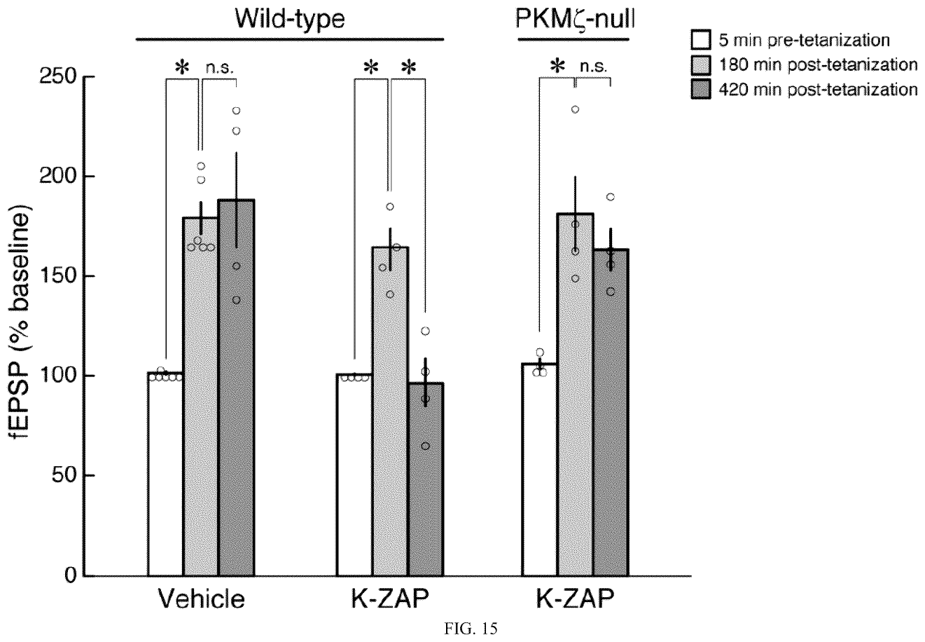

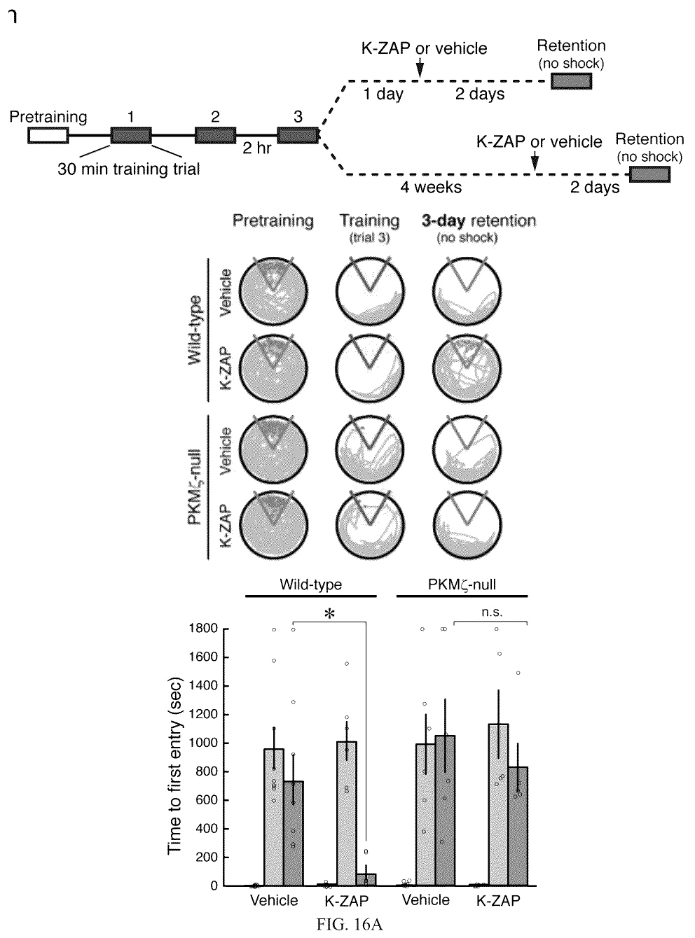

To further test that blocking KIBRA-PKMζ interaction is an effective way to reverse LTP and memory, we inhibited KIBRA-PKMζ dimerization using a cell-permeable, myristoylated peptide that mimics KIBRA's PKMζ-anchoring sequence (28) (myr-N-FVRNSLERRSVRMKRPS-C, referred to herein as KIBRA-based zeta antagonist peptide, K-ZAP, FIGS. 14, A and B). K-ZAP therefore is an amino-myristoylated peptide whose amino acid sequence is as set out in SEQ ID NO: 5. K-ZAP applied 3 h post-tetanization reverses late-LTP maintenance in wild-type mice, with no measurable effect on baseline synaptic transmission (FIG. 14C and FIG. 15). Like ζ-stat, K-ZAP has no effect on late-LTP maintenance in PKMζ-null mice. Intrahippocampal injection of K-ZAP 1 day after active place avoidance conditioning disrupts established spatial long-term memory retention assayed 2 days post-injection in wild-type mice (FIG. 16A). K-ZAP has no effect on memory retention in PKMζ-null mice.

### Example 5: Persistent KIBRA-PKMζ Coupling Maintains Remote Memory

We examined whether KIBRA-PKMζ interaction maintains memory despite PKMζ turnover. PKMζ turns over within a few hours in cultured hippocampal neurons (27) and within days in hippocampus in vivo, as indicated by the loss of PKMζ protein after shRNA knockdown of PKMζ mRNA in wild-type animals (24, 25) or inducible deletion of the PKMζ gene in Prkcz^{fl/fl} mice (FIG. 17). Although PKMζ molecules are replaced, active place avoidance conditioning of wild-type mice produces a stable persistent increase in the steady-state level of PKMζ in hippocampus that lasts for over 4 weeks, and the conditioned behavior remains hippocampus-dependent in the face of systems consolidation (7, 19). We therefore trained mice on active place avoidance and 4 weeks later injected either K-ZAP or vehicle bilaterally in hippocampus (FIG. 16B). Retention testing 2 days after injection reveals K-ZAP disrupts remote spatial memory.

This foregoing examples of interference with memory by administering K-ZAP as disclosed herein is surprising in view of prior report showing that intrahippocampal administration of an adeno-associated vector driving expression of a PKMζ-binding fragment of KIBRA, corresponding to amino acids 956-975 of KIBRA (and therefore including a peptide that prevents KIBRA-PKMζ binding as disclosed herein) improved memory performance in a rotating disc spatial avoidance paradigm, and that lentiviral vector-mediated intrahippocampal delivery of a C-terminal fragment of KIBRA, including a PKMζ-binding domain as disclosed herein, improves memory in an object-context discrimination test in a mouse tauopathy model. See Vogt-Eisele et al., 2014, KIBRA (KIdney/BRAin protein) regulates learning and memory and stabilizes Protein kinase MG. J Neurochem. 2014, 128 (5): 686-700; Kauwe et al., 2024, KIBRA repairs synaptic plasticity and promotes resilience to tauopathy-related memory loss, J Clin Invest. 2024; 134 (3): e169064. Thus, as disclosed herein, the lipopeptide K-ZAP expressed a surprising ability to disrupt memory- and LTP-related processes following in vivo administration, as may be clinically relevant for treatment of anxiety, depression, addiction, neuropathic pain, r related disorders as disclosed herein.

Although some non-limiting examples have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

### DISCUSSION

As disclosed herein, coupling of PKMζ and KIBRA is necessary for long-term memory maintenance, providing a mechanism that also addresses the general question of how an increase/activation of kinase signaling specifically targets action only at activated synapses. Interaction between PKMζ and KIBRA may maintain LTP and memory (FIGS. 7, 9, 10, 12, 14, and 16).

As disclosed herein, Synaptic stimulation that induces LTP facilitates the formation of KIBRA-PKMζ complexes that persist at least 3 hours in late-LTP maintenance (FIG. 1), and functional KIBRA-PKMζ coupling maintains late-LTP in hippocampal slices and memory lasting weeks in vivo (FIGS. 7, 9, 10, 12, 14, and 16). This persistent coupling of KIBRA and PKMζ contrasts with the activities of other molecules that last for only seconds to minutes after strong synaptic stimulation, including CaMKII, cyclic AMP-dependent protein kinase (PKA), and most PKC isoforms (11, 43, 44), as well as local modulators of actin dynamics (45) and activators of gene expression that increase the synthesis of proteins cell-wide (42, 52). These signaling molecules also act transiently for only minutes to hours during initial cellular memory consolidation (44, 45, 52), as compared to the persistent action of KIBRA-PKMζ coupling necessary for memory maintenance lasting weeks (FIG. 16B). Blocking PKMζ with antisense-oligodeoxynucleotides or shRNA shows no effect on learning or initial short-term forms of memory, while preventing long-term memory (17, 25). Therefore, coupling PKMζ action to KIBRA is likely not essential for initial, transient forms of memory. KIBRA-PKMζ coupling, however, could play a role in the initiation of late-LTP and long-term memory, and this important question requires investigating the coupling during the formation of these fundamental processes.

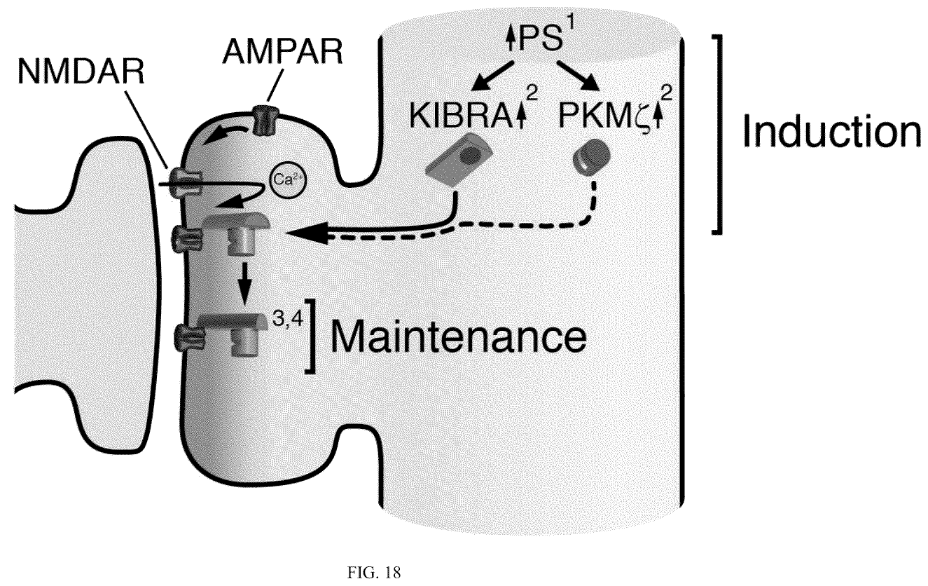

Without being limited to any particular mechanism of action, we propose that KIBRA may act as a synaptic tag aligning PKMζ at activated synapses (FIG. 18). KIBRA interacts with postsynaptic α-amino-3-hydroxy-5-methyl-4-isoxazolepropionic acid receptors (AMPARs), functioning as a scaffolding protein to regulate trafficking of the receptors (38). Blocking PKMζ from binding the KIBRA tag during and after tetanization prevents the formation of late-LTP, allowing for early-LTP (FIG. 9B). Therefore, increased number of postsynaptic AMPARs in early-LTP may sequester KIBRA at activated synapses, and the scaffolding protein then acts as a tag at these synapses to anchor PKMζ (FIG. 18). In LTP maintenance, both KIBRA and PKMζ are present in CA1 pyramidal cell somata, but they do not seem to be in a complex (FIG. 1B and FIG. 3A).

As disclosed herein, once established, the continual alignment of KIBRA and PKMζ maintains late-LTP and long-term memory (FIG. 18). Antagonists that block the KIBRA-binding site in PKMζ or mimic the PKMζ-binding site in KIBRA reverse late-LTP at activated synapses when applied 3 h after tetanization and have no measurable effect on unactivated, resting synaptic pathways in the same slice preparation (FIG. 7A and FIG. 14C) or on baseline synaptic transmission (FIG. 9B). These data support the specificity of the potentiating effects of KIBRA-PKMζ coupling in activated synapses. The exclusive action of KIBRA-PKMζ inhibitors on late-LTP maintenance contrasts with CaMKII inhibitors, which either block LTP induction but not maintenance (57-59), or affect LTP within an hour of tetanization (60). In spatial memory, PKMζ increases selectively in subpopulations of neurons active during memory formation (marked by Arc-promoter activation) and in subsets of synaptic spines of these active neurons (19).

Both antagonists of KIBRA-PKMζ coupling erase established late-LTP and long-term memory (FIG. 7A, and FIG. 9A, and FIG. 10, and FIG. 12, and FIG. 14C, and FIG. 16, and FIG. 11); however, neither antagonist affects LTP or memory in PKMζ-null mice (FIG. 7B, and FIG. 10, and FIG. 12, and FIG. 14C, and FIG. 16A). The PKMζ-independent maintenance of these mutant mice and also wild-type mice (FIG. 13) could be through the prolonged actions of other PKCs (17), CaMKII (60), or other molecular mechanisms (49, 50, 62-65). The transient increase of PKCι/λ during early-LTP in wild-type mice becomes persistent during late-LTP in PKMζ-null mice (17). Whether KIBRA anchors and stabilizes PKCι/λ at activated synapses after genetic deletion of PKMζ remains to be explored, but could provide a parsimonious account for compensation in PKMζ-null mutant mice. An initial, weak PKCι/λ interaction with KIBRA (FIG. 4B), which is displaced by the strong interaction of newly synthesized PKMζ in wild-type mice, might persist in the absence of PKMζ. Indeed, a PKCι/λ inhibitor disrupts late-LTP and long-term memory maintenance exclusively in PKMζ-null mice and not wild-type mice (17). These pharmacogenetic experiments with ζ- and ι/λ-antagonists support: 1) the KIBRA-PKMζ maintenance hypothesis that late-LTP and long-term memory in wild-type mice share a common PKMζ-dependent molecular mechanism of information storage (7), and 2) mutant mice lacking PKMζ recruit compensatory PKMζ-independent maintenance mechanisms (17).

As disclosed herein, the persistent coupling of KIBRA to PKMζ's potentiating action at activated synapses maintains memory longer than the predicted lifespans of individual KIBRA (28, 39, 40) and PKMζ molecules (24, 25) (FIG. 16B and FIG. 17). Thus, as in Crick's hypothesis, the molecules of KIBRA and PKMζ must be replaced with new molecules, and these newly synthesized proteins must be targeted to appropriate sites at activated synapses. The degrading KIBRA and PKMζ are likely exchanged by new molecules produced by local dendritic synthesis from PKMζ mRNA (15), and perhaps KIBRA mRNA, as well (67). Indeed, a positive feedback loop by which PKMζ upregulates dendritic synthesis, including that from its own mRNA, has been reported (68, 69). KIBRA anchoring PKMζ's action may further localize this dendritic synthesis to activated synapses.

The antagonists that block formation of KIBRA-PKMζ complexes (FIG. 4 and FIGS. 14, A and B) reverse established late-LTP within a few hours of their application (FIG. 7A, and FIG. 9A, and FIG. 14C). Therefore, the exchange of complexes within activated synapses may be rapid. Both KIBRA (28, 39, 40) and PKMζ (24, 25) (FIG. 17) appear from knockdown experiments to turn over in neurons within hours to days, while KIBRA-PKMζ interactions sustain memory for at least 4 weeks (FIG. 16B). Thus, long-term memory may be maintained by continual exchange of potentiating molecules at activated synapses, a concept we call “persistent synaptic tagging.”

SEQ ID Nos:
SEQ ID NO: 1:
FVRNSLERRSVRM.
SEQ ID NO: 2:
PPFVRNSLERRSVRM
SEQ ID NO: 3:
FVRNSLERRSVRMKR
SEQ ID NO: 4:
DSSTLSKKPPFVRNSLERRSVRMKRP
SPPPQ
SEQ ID NO: 5:
FVRNSLERRSVRMKRPS
SEQ ID NO: 6:
X₉₅₈X₉₅₉X₉₆₀X₉₆₁X₉₆₂X₉₆₃X₉₆₄X₉₆₅
X₉₆₆X₉₆₇X₉₆₈X₉₆₉X₉₇₀

wherein X₉₅₈ through X₉₇₀ comprise acids amino acids FVRNSLERRSVRM, respectively, except that one or more of X₉₅₈ may be A or any amino acid other than F, X₉₅₉ may be A or any amino acid other than V, X₉₆₀ may be A or any amino acid other than R, X₉₆₁ may be A or any amino acid other than N, X₉₆₂ may be A or any amino acid other than S, X₉₆₃ may be A or any amino acid other than L, X₉₆₄ may be A or any amino acid other than E, X₉₆₆ may be A or any amino acid other than R, X₉₆₇ may be A or any amino acid other than S, X₉₆₈ may be A or any amino acid other than V, X₉₆₉ may be A or any amino acid other than R, and X₉₇₀ may be A or any amino acid other than M

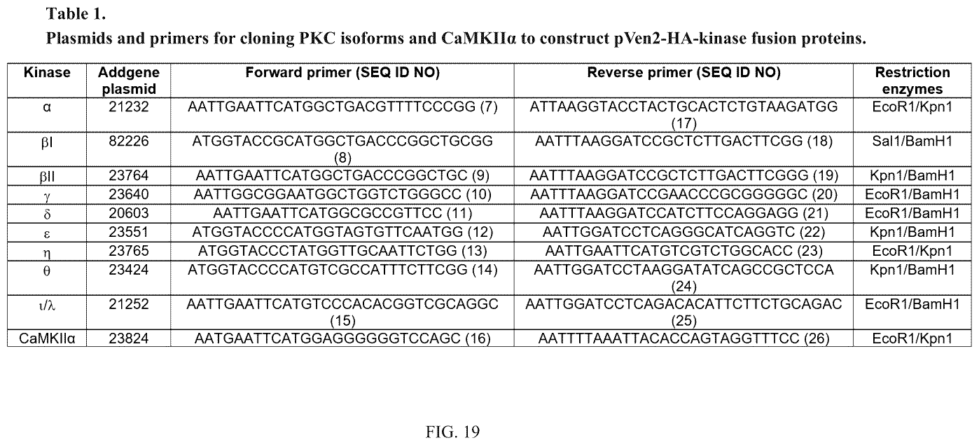

SEQ ID NOs. 7-26:
See FIG. 19
SEQ ID NO: 27
RQIKIWFQNRRMKWKK
SEQ ID NO: 28
GRKKRRORRRPPQ
SEQ ID NO: 29
AGYLLGKINLKALAALAKKIL
SEQ ID NO: 30
RRRRRRRRRRR
SEQ ID NO: 31
RIFIHFRQGQ
SEQ ID NO: 32
RIFIHFRQGC
SEQ ID NO: 33
RIFIHFRIGQ
SEQ ID NO: 34
RIFIHFRIGC

### REFERENCES

1. F. Crick, Memory and molecular turnover. Nature 312, 101 (1984).
2. J. E. Lisman, A mechanism for memory storage insensitive to molecular turnover: a bistable autophosphorylating kinase. Proc Natl Acad Sci USA 82, 3055-3057 (1985).
3. S. J. Martin, P. D. Grimwood, R. G. Morris, Synaptic plasticity and memory: an evaluation of the hypothesis. Annu Rev Neurosci 23, 649-711 (2000).
4. T. V. P. Bliss, T. Lomo, Long-lasting potentiation of synaptic transmission in the dentate area of the anaesthetized rabbit following stimulation of the perforant path. J. Physiol. 232, 331-356 (1973).
5. D. S. Ling, L. S. Benardo, P. A. Serrano, N. Blace, M. T. Kelly, J. F. Crary, T. C. Sacktor, Protein kinase Mζ is necessary and sufficient for LTP maintenance. Nat Neurosci 5, 295-296 (2002).
6. D. S. Ling, L. S. Benardo, T. C. Sacktor, Protein kinase M enhances excitatory synaptic transmission by increasing the number of active postsynaptic AMPA receptors. Hippocampus 16, 443-452 (2006).
7. E. Pastalkova, P. Serrano, D. Pinkhasova, E. Wallace, A. A. Fenton, T. C. Sacktor, Storage of spatial information by the maintenance mechanism of LTP. Science 313, 1141-1144 (2006).
8. T. C. Sacktor, How does PKMζ maintain long-term memory? Nat Rev Neurosci 12, 9-15 (2011).
9. P. W. Frankland, S. A. Josselyn, Neuroscience: Memory and the single molecule. Nature 493, 312-313 (2013).
10. T. C. Sacktor, A. A. Fenton, What does LTP tell us about the roles of CaMKII and PKMzeta in memory? Molecular brain 11, 77 (2018).
11. T. C. Sacktor, P. Osten, H. Valsamis, X. Jiang, M. U. Naik, E. Sublette, Persistent activation of the (isoform of protein kinase C in the maintenance of long-term potentiation. Proc Natl Acad Sci USA 90, 8342-8346 (1993).
12. A. I. Hernandez, N. Blace, J. F. Crary, P. A. Serrano, M. Leitges, J. M. Libien, G. Weinstein, A. Tcherapanov, T. C. Sacktor, Protein kinase M synthesis from a brain mRNA encoding an independent protein kinase CE catalytic domain. Implications for the molecular mechanism of memory. J Biol Chem 278, 40305-40316 (2003).
13. H. Oster, G. Eichele, M. Leitges, Differential expression of atypical PKCs in the adult mouse brain. Brain research. Molecular brain research 127, 79-88 (2004).
14. A. I. Hernandez, W. C. Oxberry, J. F. Crary, S. S. Mirra, T. C. Sacktor, Cellular and subcellular localization of PKMzeta. Philosophical transactions of the Royal Society of London. Series B. Biological sciences 369, 20130140 (2014).
15. I. A. Muslimov, V. Nimmrich, A. I. Hernandez, A. Tcherepanov, T. C. Sacktor, H. Tiedge, Dendritic transport and localization of protein kinase Mζ mRNA: Implications for molecular memory consolidation. J Biol Chem 279, 52613-52622 (2004).
16. P. Osten, L. Valsamis, A. Harris, T. C. Sacktor, Protein synthesis-dependent formation of protein kinase Mζ in long-term potentiation. J Neurosci 16, 2444-2451 (1996).
17. P. Tsokas, C. Hsieh, Y. Yao, E. Lesburgueres, E. J. Wallace, A. Tcherepanov, D. Jothianandan, B. R. Hartley, L. Pan, B. Rivard, R. V. Farese, M. P. Sajan, P. J. Bergold, A. I. Hernandez, J. E. Cottrell, H. Z. Shouval, A. A. Fenton, T. C. Sacktor, Compensation for PKMzeta in long-term potentiation and spatial long-term memory in mutant mice. eLife 5, e14846 (2016).
18. C. Hsieh, P. Tsokas, P. Serrano, A. I. Hernandez, D. Tian, J. E. Cottrell, H. Z. Shouval, A. A. Fenton, T. C. Sacktor, Persistent increased PKMzeta in long-term and remote spatial memory. Neurobiology of learning and memory 138, 135-144 (2017).
19. C. Hsieh, P. Tsokas, A. Grau-Perales, E. Lesburgueres, J. Bukai, K. Khanna, J. Chorny, A. Chung, C. Jou, N. S. Burghardt, C. A. Denny, R. E. Flores-Obando, B. R. Hartley, L. M. Rodriguez Valencia, A. I. Hernandez, P. J. Bergold, J. E. Cottrell, J. M. Alarcon, A. A. Fenton, T. C. Sacktor, Persistent increases of PKMzeta in memory-activated neurons trace LTP maintenance during spatial long-term memory storage. Eur J Neurosci 54, 6795-6814 (2021).
20. Y. Hara, M. Punsoni, F. Yuk, C. S. Park, W. G. Janssen, P. R. Rapp, J. H. Morrison, Synaptic distributions of GluA2 and PKMzeta in the monkey dentate gyrus and their relationships with aging and memory. J Neurosci 32, 7336-7344 (2012).
21. P. P. Gao, J. H. Goodman, T. C. Sacktor, J. T. Francis, Persistent increases of PKMζ in sensorimotor cortex maintain procedural long-term memory storage. iScience 5, 90-98 (2018).
22. L. J. Volk, J. L. Bachman, R. Johnson, Y. Yu, R. L. Huganir, PKM-zeta is not required for hippocampal synaptic plasticity, learning and memory. Nature 493, 420-423 (2013).
23. A. M. Lee, B. R. Kanter, D. Wang, J. P. Lim, M. E. Zou, C. Qiu, T. McMahon, J. Dadgar, S. C. Fischbach-Weiss, R. O. Messing, Prkcz null mice show normal learning and memory. Nature 493, 416-419 (2013).
24. Z. Dong, H. Han, H. Li, Y. Bai, W. Wang, M. Tu, Y. Peng, L. Zhou, W. He, X. Wu, T. Tan, M. Liu, X. Wu, W. Zhou, W. Jin, S. Zhang, T. C. Sacktor, T. Li, W. Song, Y. T. Wang, Long-term potentiation decay and memory loss are mediated by AMPAR endocytosis. The Journal of clinical investigation 125, 234-247 (2015).
25. S. Wang, T. Sheng, S. Ren, T. Tian, W. Lu, Distinct Roles of PKCiota/lambda and PKMzeta in the Initiation and Maintenance of Hippocampal Long-Term Potentiation and Memory. Cell Rep 16, 1954-1961 (2016).
26. A. Pavlowsky, E. Wallace, A. A. Fenton, J. M. Alarcon, Persistent modifications of hippocampal synaptic function during remote spatial memory. Neurobiology of learning and memory 138, 182-197 (2017).
27. S. F. Palida, M. T. Butko, J. T. Ngo, M. R. Mackey, L. A. Gross, M. H. Ellisman, R. Y. Tsien, PKMzeta, But Not PKClambda, Is Rapidly Synthesized and Degraded at the Neuronal Synapse. J Neurosci 35, 7736-7749 (2015).
28. A. Vogt-Eisele, C. Kruger, K. Duning, D. Weber, R. Spoelgen, C. Pitzer, C. Plaas, G. Eisenhardt, A. Meyer, G. Vogt, M. Krieger, E. Handwerker, D. O. Wennmann, T. Weide, B. V. Skryabin, M. Klugmann, H. Pavenstadt, M. J. Huentelmann, J. Kremerskothen, A. Schneider, KIBRA (Kidney/BRAin protein) regulates learning and memory and stabilizes Protein kinase Mzeta. Journal of neurochemistry 128, 686-700 (2013).
29. B. L. McNaughton, R. G. M. Morris, Hippocampal synaptic enhancement and information storage within a distributed memory system. Trends Neurosci 10, 409-415 (1987).
30. E. I. Moser, K. A. Krobert, M. B. Moser, R. G. Morris, Impaired spatial leaming after saturation of long-term potentiation. Science 281, 2038-2042 (1998).
31. R. Shema, S. Haramati, S. Ron, S. Hazvi, A. Chen, T. C. Sacktor, Y. Dudai, Enhancement of consolidated long-term memory by overexpression of protein kinase Mzeta in the neocortex. Science 331, 1207-1210 (2011).
32. A. Papassotiropoulos, D. A. Stephan, M. J. Huentelman, F. J. Hoerndli, D. W. Craig, J. V. Pearson, K. D. Huynh, F. Brunner, J. Corneveaux, D. Osbome, M. A. Wollmer, A. Aerni, D. Coluccia, J. Hanggi, C. R. Mondadori, A. Buchmann, E. M. Reiman, R. J. Caselli, K. Henke, D. J. de Quervain, Common Kibra alleles are associated with human memory performance. Science 314, 475-478 (2006).
33. A. Milnik, A. Heck, C. Vogler, H. J. Heinze, D. J. de Quervain, A. Papassotiropoulos, Association of KIBRA with episodic and working memory: a meta-analysis. Am J Med Genet B Neuropsychiatr Genet 159B, 958-969 (2012),
34. K. Buther, C. Plaas, A. Barnekow, J. Kremerskothen, KIBRA is a novel substrate for protein kinase Czeta. Biochemical and biophysical research communications 317, 703-707 (2004).
35. L. Zhang, S. Yang, D. O. Wennmann, Y. Chen, J. Kremerskothen, J. Dong, KIBRA: In the brain and beyond. Cellular signalling 26, 1392-1399 (2014).
36. J. Hu, L. Ferguson, K. Adler, C. A. Farah, M. H. Hastings, W. S. Sossin, S. Schacher, Selective Erasure of Distinct Forms of Long-Term Synaptic Plasticity Underlying Different Forms of Memory in the Same Postsynaptic Neuron. Current biology: CB 27, 1888-1899 e1884 (2017).
37. L. Ferguson, J. Hu, D. Cai, S. Chen, T. W. Dunn, K. Pearce, D. L. Glanzman, S. Schacher, W. S. Sossin, Isoform Specificity of PKMs during Long-Term Facilitation in Aplysia Is Mediated through Stabilization by KIBRA. J Neurosci 39, 8632-8644 (2019).
38. L. Makuch, L. Volk, V. Anggono, R. C. Johnson, Y. Yu, K. Duning, J. Kremerskothen, J. Xia, K. Takamiya, R. L. Huganir, Regulation of AMPA receptor function by the human memory-associated gene KIBRA. Neuron 71, 1022-1029 (2011).
39. F. D. Heitz, M. Farinelli, S. Mohanna, M. Kahn, K. Duning, M. C. Frey, H. Pavenstadt, I. M. Mansuy, The memory gene KIBRA is a bidirectional regulator of synaptic and structural plasticity in the adult brain. Neurobiology of learning and memory 135, 100-114 (2016).
40. M. L. Mendoza, L. D. Quigley, T. Dunham, L. J. Volk, KIBRA regulates activity-induced AMPA receptor expression and synaptic plasticity in an age-dependent manner. iScience 25, 105623 (2022).
41. O. Soderberg, M. Gullberg, M. Jarvius, K. Ridderstrale, K. J. Leuchowius, J. Jarvius, K. Wester, P. Hydbring, F. Bahram, L. G. Larsson, U. Landegren, Direct observation of individual endogenous protein complexes in situ by proximity ligation. Nat Methods 3, 995-1000 (2006).
42. U. Frey, R. G. Morris, Synaptic tagging and long-term potentiation. Nature 385, 533-536 (1997).
43. E. D. Roberson, J. D. Sweatt, Transient activation of cyclic AMP-dependent protein kinase during hippocampal long-term potentiation. J Biol Chem 271, 30436-30441 (1996).
44. R. Yasuda, Y. Hayashi, J. W. Hell, CaMKII: a central molecular organizer of synaptic plasticity, learning and memory. Nat Rev Neurosci 23, 666-682 (2022).
45. A. Goto, A. Bota, K. Miya, J. Wang, S. Tsukamoto, X. Jiang, D. Hirai, M. Murayama, T. Matsuda, T. J. McHugh, T. Nagai, Y. Hayashi, Stepwise synaptic plasticity events drive the early phase of memory consolidation. Science 374, 857-863 (2021).
46. J. Lisman, Criteria for identifying the molecular basis of the engram (CaMKII, PKMzeta). Molecular brain 10, 55 (2017).
47. P. Serrano, E. L. Friedman, J. Kenney, S. M. Taubenfeld, J. M. Zimmerman, J. Hanna, C. Alberini, A. E. Kelley, S. Maren, J. W. Rudy, J. C. Yin, T. C. Sacktor, A. A. Fenton, PKMζ maintains spatial, instrumental, and classically conditioned long-term memories. PLOS Biol 6, 2698-2706 (2008).
48. J. L. Kwapis, T. J. Jarome, M. E. Lonergan, F. J. Helmstetter, Protein kinase Mzeta maintains fear memory in the amygdala but not in the hippocampus. Behavioral neuroscience 123, 844-850 (2009).
49. K. Si, E. R. Kandel, The Role of Functional Prion-Like Proteins in the Persistence of Memory. Cold Spring Harb Perspect Biol 8, a021774 (2016).
50. R. Y. Tsien, Very long-term memories may be stored in the pattern of holes in the perineuronal net. Proc Natl Acad Sci USA 110, 12456-12461 (2013).
51. S. Heo, G. H. Diering, C. H. Na, R. S. Nirujogi, J. L. Bachman, A. Pandey, R. L. Huganir, Identification of long-lived synaptic proteins by proteomic analysis of synaptosome protein turnover. Proc Natl Acad Sci USA 115, E3827-E3836 (2018).
52. P. Shrestha, E. Klann, Spatiotemporally resolved protein synthesis as a molecular framework for memory consolidation. Trends Neurosci 45, 297-311 (2022).
53. Y. Yao, M. T. Kelly, S. Sajikumar, P. Serrano, D. Tian, P. J. Bergold, J. U. Frey, T. C. Sacktor, PKMζ maintains late long-term potentiation by N-ethylmaleimide-sensitive factor/GluR2-dependent trafficking of postsynaptic AMPA receptors. J Neurosci 28, 7820-7827 (2008).
54. J. G. Hanley, L. Khatri, P. I. Hanson, E. B. Ziff, NSF ATPase and alpha-/beta-SNAPs disassemble the AMPA receptor-PICK1 complex. Neuron 34, 53-67 (2002).
55. P. V. Migues, O. Hardt, D. C. Wu, K. Gamache, T. C. Sacktor, Y. T. Wang, K. Nader, PKMζ maintains memories by regulating GluR2-dependent AMPA receptor trafficking. Nat Neurosci 13, 630-634 (2010).
56. D. O. Wennmann, J. Schmitz, M. C. Wehr, M. P. Krahn, N. Koschmal, S. Gromnitza, U. Schulze, T. Weide, A. Chekuri, B. V. Skryabin, V. Gerke, H. Pavenstadt, K. Duning, J. Kremerskothen, Evolutionary and molecular facts link the WWC protein family to Hippo signaling. Mol Biol Evol 31, 1710-1723 (2014).
57. N. Otmakhov, L. C. Griffith, J. E. Lisman, Postsynaptic inhibitors of calcium/calmodulin-dependent protein kinase type II block induction but not maintenance of pairing-induced long-term potentiation. J Neurosci 17, 5357-5365 (1997).
58. I. Buard, S. J. Coultrap, R. K. Freund, Y. S. Lee, M. L. Dell′Acqua, A. J. Silva, K. U. Bayer, CaMKII “autonomy” is required for initiating but not for maintaining neuronal long-term information storage. J Neurosci 30, 8214-8220 (2010).
59. H. Murakoshi, M. E. Shin, P. Parra-Bueno, E. M. Szatmari, A. C. Shibata, R. Yasuda, Kinetics of Endogenous CaMKII Required for Synaptic Plasticity Revealed by Optogenetic Kinase Inhibitor. Neuron 94, 37-47 e35 (2017).
60. W. Tao, J. Lee, X. Chen, J. Diaz-Alonso, J. Zhou, S. Pleasure, R. A. Nicoll, Synaptic memory requires CaMKII. Elife 10, (2021).
61. J. H. Choi, S. E. Sim, J. I. Kim, D. I. Choi, J. Oh, S. Ye, J. Lee, T. Kim, H. G. Ko, C. S. Lim, B. K. Kaang, Interregional synaptic maps among engram cells underlie memory formation. Science 360, 430-435 (2018).
62. T. J. Ryan, D. S. Roy, M. Pignatelli, A. Arons, S. Tonegawa, Memory. Engram cells retain memory under retrograde amnesia. Science 348, 1007-1013 (2015).
63. W. Aziz, I. Kraev, K. Mizuno, A. Kirby, T. Fang, H. Rupawala, K. Kasbi, S. Rothe, F. Jozsa, K. Rosenblum, M. G. Stewart, K. P. Giese, Multi-input Synapses, but Not LTP-Strengthened Synapses, Correlate with Hippocampal Memory Storage in Aged Mice. Current biology: CB 29, 3600-3610 e3604 (2019),
64. W. C. Abraham, O. D. Jones, D. L. Glanzman, Is plasticity of synapses the mechanism of long-term memory storage? NPJ Sci Learn 4, 9 (2019).
65. A. I. Ramsaran, Y. Wang, A. Golbabaei, S. Aleshin, M. L. de Snoo, B. A. Yeung, A. J. Rashid, A. Awasthi, J. Lau, L. M. Tran, S. Y. Ko, A. Abegg, L. C. Duan, C. Mckenzie, J. Gallucci, M. Ahmed, R. Kaushik, A. Dityatev, S. A. Josselyn, P. W. Frankland, A shift in the mechanisms controlling hippocampal engram formation during brain maturation. Science 380, 543-551 (2023).
66. G. Kauwe, K. A. Pareja-Navarro, L. Yao, J. H. Chen, I. Wong, R. Saloner, H. Cifuentes, A. L. Nana, S. Shah, Y. Li, D. Le, S. Spina, L. T. Grinberg, W. W. Seeley, J. H. Kramer, T. C. Sacktor, B. Schilling, L. Gan, K. B. Casaletto, T. E. Tracy, KIBRA repairs synaptic plasticity and promotes resilience to tauopathy-related memory loss. The Journal of clinical investigation 134, (2024).
67. I. J. Cajigas, G. Tushev, T. J. Will, S. tom Dieck, N. Fuerst, E. M. Schuman, The local transcriptome in the synaptic neuropil revealed by deep sequencing and high-resolution imaging. Neuron 74, 453-466 (2012).
68. M. T. Kelly, J. F. Crary, T. C. Sacktor, Regulation of protein kinase Mζ synthesis by multiple kinases in long-term potentiation. J Neurosci 27, 3439-3444 (2007).
69. P. R. Westmark, C. J. Westmark, S. Wang, J. Levenson, K. J. O'Riordan, C. Burger, J. S. Malter, Pin1 and PKMzeta sequentially control dendritic protein synthesis. Sci Signal 3, ra18 (2010).
70. R. L. Redondo, R. G. Morris, Making memories last: the synaptic tagging and capture hypothesis. Nat Rev Neurosci 12, 17-30 (2011).
71. Y. Kodama, C. D. Hu, Bimolecular fluorescence complementation (BiFC): a 5-year update and future perspectives. Biotechniques 53, 285-298 (2012).
72. Plutarch, “Theseus” in Lives, Volume I: Theseus and Romulus. Lycurgus and Numa. Solon and Publicola. Translated by Bernadotte Perrin. (Loeb Classical Library, Harvard University Press, Cambridge, MA) Chapter 23.1. (1914).
73. P. Tsokas, B. Rivard, C. Hsieh, J. E. Cottrell, A. A. Fenton, T. C. Sacktor, Antisense Oligodeoxynucleotide Perfusion Blocks Gene Expression of Synaptic Plasticity-related Proteins without Inducing Compensation in Hippocampal Slices. Bio Protoc 9, (2019).
74. P. Tsokas, E. A. Grace, P. Chan, T. Ma, S. C. Sealfon, R. Iyengar, E. M. Landau, R. D. Blitzer, Local protein synthesis mediates a rapid increase in dendritic elongation factor 1A after induction of late long-term potentiation. J Neurosci 25, 5833-5843 (2005).
75. W. W. Anderson, G. L. Collingridge, Capabilities of the WinLTP data acquisition program extending beyond basic LTP experimental functions. J Neurosci Methods 162, 346-356 (2007).
76. T. Peng, K. Thorn, T. Schroeder, L. Wang, F. J. Theis, C. Marr, N. Navab, A BasiC tool for background and shading correction of optical microscopy images. Nat Commun 8, 14836 (2017).
77. T. D. Goddard, C. C. Huang, E. C. Meng, E. F. Pettersen, G. S. Couch, J. H. Morris, T. E. Ferrin, UCSF ChimeraX: Meeting modern challenges in visualization and analysis. Protein Sci 27, 14-25 (2018).
78. E. F. Pettersen, T. D. Goddard, C. C. Huang, E. C. Meng, G. S. Couch, T. I. Croll, J. H. Morris, T. E. Ferrin, UCSF ChimeraX: Structure visualization for researchers, educators, and developers. Protein Sci 30, 70-82 (2021).
79. U. Pieper, N. Eswar, H. Braberg, M. S. Madhusudhan, F. P. Davis, A. C. Stuart, N. Mirkovic, A. Rossi, M. A. Marti-Renom, A. Fiser, B. Webb, D. Greenblatt, C. C. Huang, T. E. Ferrin, A. Sali, MODBASE, a database of annotated comparative protein structure models, and associated resources. Nucleic Acids Res 32, D217-222 (2004).
80. C. A. Schneider, W. S. Rasband, K. W. Eliceiri, NIH Image to ImageJ: 25 years of image analysis. Nat Methods 9, 671-675 (2012).
81. A. M. Delachat, N. Guidotti, A. L. Bachmann, A. C. A. Meireles-Filho, H. Pick, C. C. Lechner, C. Deluz, B. Deplancke, D. M. Suter, B. Fierz, Engineered Multivalent Sensors to Detect Coexisting Histone Modifications in Living Stem Cells. Cell Chem Biol 25, 51-56 e56 (2018).
82. A. Garcia-Osta, P. Tsokas, G. Pollonini, E. M. Landau, R. Blitzer, C. M. Alberini, MuSK expressed in the brain mediates cholinergic responses, synaptic plasticity, and memory formation. J Neurosci 26, 7919-7932 (2006).
83. E. M. M. Manders, F. J. Verbeek, J. A. Aten, Measurement of co-localization of objects in dual-colour confocal images. J Microscopy 169, 375-382 (1993).

1. A method of disrupting memory, comprising
administering to a subject a peptide, wherein the peptide prevents biding of kidney and brain expressed protein (KIBRA) to protein kinase M zeta (PKMzeta), and the peptide comprises amino acid sequence X₉₅₈X₉₅₉X₉₆₀X₉₆₁X₉₆₂X₉₆₃X₉₆₄X₉₆₅X₉₆₆X₉₆₇X₉₆₈X₉₆₉X₉₇₀, wherein X₉₅₈ through X₉₇₀ comprise acids amino acids FVRNSLERRSVRM, respectively, except that X₉₅₈ may be any amino acid other than F, X₉₅₉ may be any amino acid other than V, X₉₆₀ may be any amino acid other than R, X₉₆₁ may be any amino acid other than N, X₉₆₂ may be any amino acid other than S, X₉₆₃ may be any amino acid other than L, X₉₆₄ may be any amino acid other than E, X₉₆₅ is R, X₉₆₆ may be any amino acid other than R, X₉₆₇ may be any amino acid other than S, X₉₆₈ may be any amino acid other than V, X₉₆₉ may be any amino acid other than R, or X₉₇₀ may be any amino acid other than M.

2. The method of claim 1, wherein X₉₅₈ may be A, X₉₅₉ may be A, X₉₆₀ may be A, X₉₆₁ may be A, X₉₆₂ may be A, X₉₆₃ may be A, X₉₆₄ may be A, X₉₆₆ may be A, X₉₆₇ may be A, X₉₆₈ may be A, X₉₆₉ may be A, or X₉₇₀ may be A.

3. The method of claim 1, wherein the amino acid sequence is X₉₅₆X₉₅₇X₉₅₈FVRNSLERRSVRM, wherein X₉₅₆ may be A or any amino acid other than P, or X₉₅₇ may be A or any amino acid other than P.

4. The method of claim 1, wherein the amino acid sequence is DSSTLSKKX₉₅₆X₉₅₇X₉₅₈X₉₅₉X₉₆₀X₉₆₁X₉₆₂X₉₆₃X₉₆₄X₉₆₅X₉₆₆X₉₆₇X₉₆₈X₉₆₉X₉₇₀KRPSPPPQ.

5. (canceled)

6. The method of claim 1, wherein the peptide further comprises a cell-penetrating peptide sequence.

7. The method of claim 1, wherein the peptide further comprises a cell-penetrating peptide sequence and the cell-penetrating peptide sequence is selected from SEQ ID NO: 27, SEQ ID NO: 28, SEQ ID NO: 29, and SEQ ID NO: 30.

8. The method of claim 1, wherein the peptide further comprises a fatty acyl group, wherein the fatty acyl group is selected from a C12 to a C18 fatty acid.

9. The method of claim 1, wherein the peptide further comprises an N-terminal myristoylation.

10. The method of claim 1, wherein the administering comprises administering a vector and the vector contains the peptide.

11. The method of claim 1, wherein the administering comprises intracerebral injection.

12. The method of claim 1, wherein the administering is selected from septohippocampal administration, amygdalar administration, cerebral cortical administration, spinal cord administration, striatal administration, and cerebellar administration.

13. (canceled)

14. The method of claim 1, wherein the amino acid sequence is SEQ ID NO: 1.

15. The method of claim 1, wherein the amino acid sequence is SEQ ID NO: 2.

16. The method of claim 1, wherein the amino acid sequence is SEQ ID NO: 3.

17. The method of claim 1, wherein the amino acid sequence is SEQ ID NO: 4.

18. The method of claim 1, wherein the amino acid sequence is SEQ ID NO: 5.

19. The method of claim 1, wherein administering comprises administering to a subject in need of treatment for an addition.

20. The method of claim 1, wherein administering comprises administering to a subject in need of treatment for neuropathic pain.

21. The method of claim 1, wherein administering comprises administering to a subject in need of treatment for an anxiety disorder.

22. A peptide, wherein the peptide prevents biding of kidney and brain expressed protein (KIBRA) to protein kinase M zeta (PKMzeta), the peptide comprises amino acid sequence X₉₅₈X₉₅₉X₉₆₀X₉₆₁X₉₆₂X₉₆₃X₉₆₄X₉₆₅X₉₆₆X₉₆₇X₉₆₈X₉₆₉X₉₇₀, wherein X₉₅₈ through X₉₇₀ comprise acids amino acids FVRNSLERRSVRM, respectively, except that X₉₅₈ may be any amino acid other than F, X₉₅₉ may be any amino acid other than V, X₉₆₀ may be any amino acid other than R, X₉₆₁ may be any amino acid other than N, X₉₆₂ may be any amino acid other than S, X₉₆₃ may be any amino acid other than L, X₉₆₄ may be any amino acid other than E, X₉₆₅ is R, X₉₆₆ may be any amino acid other than R, X₉₆₇ may be any amino acid other than S, X₉₆₈ may be any amino acid other than V, X₉₆₉ may be any amino acid other than R, or X₉₇₀ may be any amino acid other than M, and wherein the peptide further comprises one or both of a cell-penetrating peptide sequence or a fatty acyl group, wherein the fatty acyl group is from a C12 to a C18 fatty acid.

23-36. (canceled)


SEQUENCE LISTING (34 sequences)

SEQ ID NO: 1 - protein, 13 aa, synthetic construct
FVRNSLERRSVRM

SEQ ID NO: 2 - protein, 15 aa, synthetic construct
PPFVRNSLERRSVRM

SEQ ID NO: 3 - protein, 15 aa, synthetic construct
FVRNSLERRSVRMKR

SEQ ID NO: 4 - protein, 31 aa, synthetic construct
DSSTLSKKPPFVRNSLERRSVRMKRPSPPPQ

SEQ ID NO: 5 - protein, 17 aa, synthetic construct
FVRNSLERRSVRMKRPS

SEQ ID NO: 6 - skipped

SEQ ID NO: 7 - DNA, 29 bp, synthetic construct
  source 1..29: mol_type = other DNA
aattgaattcatggctgacgttttcccgg

SEQ ID NO: 8 - DNA, 29 bp, synthetic construct
  source 1..29: mol_type = other DNA
atggtaccgcatggctgacccggctgcgg

SEQ ID NO: 9 - DNA, 27 bp, synthetic construct
  source 1..27: mol_type = other DNA
aattgaattcatggctgacccggctgc

SEQ ID NO: 10 - DNA, 26 bp, synthetic construct
  source 1..26: mol_type = other DNA
aattggcggaatggctggtctgggcc

SEQ ID NO: 11 - DNA, 23 bp, synthetic construct
  source 1..23: mol_type = other DNA
aattgaattcatggcgccgttcc

SEQ ID NO: 12 - DNA, 27 bp, synthetic construct
  source 1..27: mol_type = other DNA
atggtaccccatggtagtgttcaatgg

SEQ ID NO: 13 - DNA, 26 bp, synthetic construct
  source 1..26: mol_type = other DNA
atggtaccctatggttgcaattctgg

SEQ ID NO: 14 - DNA, 28 bp, synthetic construct
  source 1..28: mol_type = other DNA
atggtaccccatgtcgccatttcttcgg

SEQ ID NO: 15 - DNA, 31 bp, synthetic construct
  source 1..31: mol_type = other DNA
aattgaattcatgtcccacacggtcgcaggc

SEQ ID NO: 16 - DNA, 26 bp, synthetic construct
  source 1..26: mol_type = other DNA
aatgaattcatggaggggggtccagc

SEQ ID NO: 17 - DNA, 31 bp, synthetic construct
  source 1..31: mol_type = other DNA
attaaggtacctactgcactctgtaagatgg

SEQ ID NO: 18 - DNA, 27 bp, synthetic construct
  source 1..27: mol_type = other DNA
aatttaaggatccgctcttgacttcgg

SEQ ID NO: 19 - DNA, 28 bp, synthetic construct
  source 1..28: mol_type = other DNA
aatttaaggatccgctcttgacttcggg

SEQ ID NO: 20 - DNA, 27 bp, synthetic construct
  source 1..27: mol_type = other DNA
aatttaaggatccgaacccgcgggggc

SEQ ID NO: 21 - DNA, 26 bp, synthetic construct
  source 1..26: mol_type = other DNA
aatttaaggatccatcttccaggagg

SEQ ID NO: 22 - DNA, 25 bp, synthetic construct
  source 1..25: mol_type = other DNA
aattggatcctcagggcatcaggtc

SEQ ID NO: 23 - DNA, 25 bp, synthetic construct
  source 1..25: mol_type = other DNA
aattgaattcatgtcgtctggcacc

SEQ ID NO: 24 - DNA, 30 bp, synthetic construct
  source 1..30: mol_type = other DNA
aattggatcctaaggatatcagccgctcca

SEQ ID NO: 25 - DNA, 32 bp, synthetic construct
  source 1..32: mol_type = other DNA
aattggatcctcagacacattcttctgcagac

SEQ ID NO: 26 - DNA, 27 bp, synthetic construct
  source 1..27: mol_type = other DNA
aattttaaattacaccagtaggtttcc

SEQ ID NO: 27 - protein, 16 aa, synthetic construct
RQIKIWFQNRRMKWKK

SEQ ID NO: 28 - protein, 13 aa, synthetic construct
GRKKRRQRRRPPQ

SEQ ID NO: 29 - protein, 21 aa, synthetic construct
AGYLLGKINLKALAALAKKIL

SEQ ID NO: 30 - protein, 11 aa, synthetic construct
RRRRRRRRRRR

SEQ ID NO: 31 - protein, 10 aa, synthetic construct
RIFIHFRQGQ

SEQ ID NO: 32 - protein, 10 aa, synthetic construct
RIFIHFRQGC

SEQ ID NO: 33 - protein, 10 aa, synthetic construct
RIFIHFRIGQ

SEQ ID NO: 34 - protein, 10 aa, synthetic construct
RIFIHFRIGC